(19) United States
(12) Patent Application Publication
(10) Pub. No.: US 2025/0066771 A1
(43) Pub. Date: Feb. 27, 2025

(54) STREAMLINED SELECTION OF NATURALLY CIRCULATING, ANTIGENIC MATCH AND HIGH-YIELD VACCINE VIRUSES FOR SEASONAL INFLUENZA VACCINE PRODUCTION

(71) Applicant: The Curators of the University of Missouri, Columbia, MO (US)

(72) Inventors: Xiufeng Henry Wan, Columbia, MO (US); Cheng Gao, Columbia, MO (US)

(21) Appl. No.: 18/792,336

(22) Filed: Aug. 1, 2024

Related U.S. Application Data
(60) Provisional application No. 63/578,043, filed on Aug. 22, 2023.

(51) Int. Cl. C12N 15/10; G16B 35/00

(52) U.S. Cl. CPC C12N 15/1089; G16B 35/00

(57) ABSTRACT

A method of predicting preferred candidate viral strains for vaccine production is described herein. A machine learning framework for predicting preferred candidate viral strains is provided that emphasizes antigen-matching and yield predictions that correlate with features of viral proteins.

### CROSS-REFERENCE TO RELATED APPLICATIONS

The present application claims priority benefits from U.S. provisional patent application Ser. No. 63/578,043 filed Aug. 22, 2023, which is hereby incorporated by reference in its entirety.

### GOVERNMENT SUPPORT STATEMENT

This invention was made with government support under A1147640, A1114433, and A1116744 awarded by the National Institutes of Health. The government has certain rights in the invention.

### FIELD OF THE DISCLOSURE

The present teachings relate to vaccines, and more particularly to a risk viral strain identification strategy that can be utilized to develop influenza vaccines that exhibit both high yield and antigenicity matching the most prevalent wild influenza strains.

### BACKGROUND OF THE DISCLOSURE

The statements in this section merely provide background information related to the present disclosure and cannot constitute prior art.

Vaccination is the primary strategy for preventing influenza vaccinations. Influenza vaccines are produced using vaccine ‘seeds,’ which are specific influenza virus strains selected as a starting material for mass vaccine production. Ideal influenza vaccine seeds possess genetic stability, high growth rates in production systems, and strong antigenic similarity to the most prevalent circulating influenza strains, also called “antigenic matching.” To achieve high antigenic match, vaccine design can look to specific influenza virus proteins. Hemagglutinin (HA) and neuraminidase (NA) are two key proteins on the surface of influenza viruses that significantly affect such virus's ability to infect host cells and spread, can undergo antigenic drift or shift. However, HA and NA can undergo antigenic drift or shift, enabling viruses to evade host immunity elicited by natural infections and or vaccinations.

Thus, annual updates of vaccine composition are necessitated to match vaccine seed virus antigenicity with that of circulating viruses. This is a time consuming process that requires global collaborative efforts coordinated through the World Health Organization (WHO) Global Influenza Surveillance and Response System. Timely selection of effective high-yield vaccine seeds is critical for seasonal influenza vaccine manufacturing. In the 2003-2004 influenza season, the predicted vaccine strain A/Fujian/411/2002-like virus recommended by WHO was unable to grow to sufficient titers in eggs causing great pressures in virus production. During the 2009 H1 N1 pandemic, vaccine supply was delayed due to the poor yield of the vaccine virus, and a global vaccine campaign was not initiated until a high-yield vaccine strain was available after the second pandemic wave.

Extant strategies for finding vaccine seeds that exhibit high stability, high yield, and high antigenic matching have significant shortcomings. The conventional strategy to achieve high yield, for example, often involves additional passages in eggs or cells, and genetic approaches rely on reassortment with a donor strain that exhibits high yield traits in eggs or cells. Both approaches may take up to 6 months as well as have limitations. Egg or cell adaptation can result in undesired antigenic changes due to additional mutations in HA and/or NA. Genetic modification strategies do not always lead to substantial improvements in yield. Therefore, selecting naturally circulating influenza vaccine strains with high-yield phenotypes directly from clinical samples, without requiring additional engineering, would be ideal and could potentially accelerate vaccine strain selection process for timely vaccine production.

Over the past few years, several computational models have been developed to identify influenza antigenic variants using genomic sequences. However, none of these models can be used to directly identify antigenic match and high-yield viruses based on genetic sequences.

Based on the foregoing, there is a need for a new approach to overcome challenges in influenza vaccine strain selection.

### BRIEF SUMMARY OF THE DISCLOSURE

Described herein is a machine learning algorithm, Machine-learning Assisted Influenza VaccinE Strain Selection framework (MAIVeSS), that enables streamlined selection of naturally circulating, antigenic match, and high-yield influenza vaccine strains directly from clinical samples by using molecular signatures of antigenicity and virus yield in the hemagglutinin (HA) of influenza A virus. Using publicly available sequences, MAIVeSS predicted potential seed viruses with antigenicity matching to the 2009 H1N1 viruses (A(H1N1)pdm09) in circulation. The wet experiments confirmed that these seed viruses grew in high yield in both cells and eggs. MAIVeSS can potentially reduce the influenza high-yield seed vaccine selection time from months to just a few days and thus facilitate timely supply of seasonal vaccines.

In one particular aspect, the present disclosure is directed to a method for identifying one or more preferred viral strains for vaccine development, said method comprising: sequencing hemagglutinin present in one or more circulating viral strains to generate one or more circulating hemagglutinin sequences; sequencing hemagglutinin present in each of one or more candidate viral strains to generate one or more candidate hemagglutinin sequences; providing an input to a machine learning algorithm, wherein the input comprises the one or more circulating hemagglutinin sequences and the one or more candidate hemagglutinin sequences; using the machine learning algorithm to predict one or more desired phenotypes selected from the group consisting of: antigenic difference values between each of the one or more circulating viral strains and each of the one or more candidate viral strains; and an egg yield value, a cell yield value, and a combined yield value for each of the one or more candidate viral strains; identifying one or more preferred viral strains from among the one or more candidate viral strains by identifying each of the one or more candidate viral strains that are predicted to have one or more desired phenotypes selected from the group consisting of: low antigenic difference values; and a high egg yield value, cell yield value, or combined yield value.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a diagrammatic outline of the creation of the machine-learning assisted MAIVeSS framework described herein for selecting high-yield vaccine seeds that match the antigenicity of circulating influenza strains.

FIGS. 2A-2C provide multiple model views of the HA of A/California/04/2009(H1 N1) (CA/04), modeled using PyMOL with protein structure from the Protein Databank (PDB) 3LZG as template. FIG. 2A provides a model view of the HA of A/California/04/2009(H1N1) (CA/04). FIG. 2B provides a side view and a top view of the model of FIG. 2A. FIG. 2C provides a front view and a back view of the model of FIG. 2A.

FIG. 3A shows an antigenic map of A(H1N1)pdm09 viruses, visualized by antigenic cartography, showing two major antigenic variant clusters.

FIG. 3B provides a visualization of the distribution of high-yield viruses within the phylogenetic tree of A(H1N1)pdm09 viruses, differentiated by virus strains that have high yield in cells only (HY^{cell}), in eggs only (HY^{egg}), and in both cells and eggs (HY^{both}).

FIG. 3C is a bar graph showing the relative quantities of two antigenically distinct variants, CA/04-like and WI/09-like, during influenza seasons from 2009 to 2020.

FIG. 3D is a bar graph showing the relative proportions of HY^{egg}, HY^{cell}, HY^{both}, and low-yield viruses in both cells and eggs across the 2009 to 2020 influenza seasons.

FIG. 3E is a bar graph showing the presence of N159K substitutions in the HA of pdmH1 N1 high yield viruses from 2009 to 2020.

FIG. 4A is a table that shows the amino acids that are located in the residues associated with antigenicity and yield properties for three A(H1 N1)pdm09 vaccines (i.e., CA/04, MI/15, and WI/19) selected by WHO and the four vaccine candidates selected by MAIVeSS.

FIG. 4B shows an antigenic map derived via antigenic cartography of A(H1N1)pdm09 viruses and the four vaccine candidates selected by MAIVeSS, wherein the positions of filled circles represent the antigenic properties derived by the HAI data with ferret antisera, and the positions of unfilled circles represent the predictive antigenic properties by MAIVeSS. Vaccine strains are marked in lighter gray, while vaccine candidates and other epidemic A(H1N1)pdm09 viruses are marked in darker gray.

FIG. 4C shows two bar graphs comparing replication efficiencies of the vaccine candidates selected by MAIVeSS.

FIG. 5A presents multiple graphs which compare the binding avidities of two A(H1 N1)pdm09 vaccines selected by WHO and the four vaccine candidates selected by MAIVeSS to the synthetic glycan analogs 3′SLN and 6′SLN.

FIG. 5B provides structural modeling of the CA/04 HA in complex with 6′SLN (PDB ID #3UBN) and with 3′SLN (PDB ID #3UBQ).

FIG. 6A shows performance metrics for machine learning models compared in antigenicity analysis experiments. Performance was measured by Root Mean Square Error (RMSE). A lower RMSE score indicates better model prediction accuracy. Bolded values indicate best performance.

FIG. 6B shows performance metrics for machine learning models compared in virus yield analysis experiments. Performance was measured by Root Mean Square Error (RMSE). A lower RMSE score indicates better model prediction accuracy. Bolded values indicate best performance.

FIG. 6C shows performance metrics for machine learning models compared in glycan binding analysis experiments. Performance was measured by Root Mean Square Error (RMSE). A lower RMSE score indicates better model prediction accuracy. Bolded values indicate best performance.

FIG. 7A shows the number of mutations discovered at specific antibody binding sites in an exemplary set of 189 mutant virus candidates.

FIG. 7B is a pie chart showing the number of mutant viruses in an exemplary set of 189 mutant viruses that had a single mutation or double, triple quadruple, quintuple, sextuple, or septuple mutations.

FIG. 7C shows the locations and biochemical properties of amino acid substitutions found in an exemplary set of 189 mutant viruses.

FIG. 8 is a heat map illustration of binding intensity of viruses to glycans on an exemplary glycan microarray. Each row represents a HA RBS mutant and each column represents and individual glycan.

FIG. 9 shows a list of 75 glycoforms printed on an exemplary glycan microarray.

FIG. 10 shows an exemplary list of 27 glycan substructures used in machine learning, grouped as terminal, internal, or basal substructures.

FIG. 11 shows biolayer interferometry analyses (BLI) for glycan binding profiling confirming the broadened binding specificity of the HY^{both} mutant D131 E-S193T-A198S.

FIG. 12 shows structural modeling based on the crystal structure of CA/04 HA complexed with 6′SLN and 3′SLN to probe the effect of N159K, K166Q, and S206T on glycan binding affinity.

FIG. 13 shows the surface sites calculated using HA protein of pdmH1 N1 virus as a template. Ratio(%) denotes the ratio of side-chain surface area to “random coil” value per residue, and a value of 20% is used as the cutoff for a residue on the HA surface area. All residues are in H3 numbering.

Corresponding reference numerals will be used throughout the several figures of the drawings.

### DETAILED DESCRIPTION OF THE DISCLOSURE

The following detailed description illustrates the claimed invention by way of example and not by way of limitation. This description will clearly enable one skilled in the art to make and use the claimed invention, and describes several embodiments, adaptations, variations, alternatives and uses of the claimed invention, including what is presently believed is the best mode of carrying out the claimed invention. Additionally, it is to be understood that the claimed invention is not limited in its applications to the details of construction and the arrangements of components set forth in the following description or illustrated in the drawings. The claimed invention is capable of other embodiments and of being practiced or being carried out in various ways. Also, it is to be understood that the phraseology and terminology used herein is for the purpose of description and should not be regarded as limiting.

The methods for identifying preferred viral strains for influenza vaccine development disclosed herein require the development of a machine-learning framework referred to herein as MAIVeSS. Thus, the following disclosure will provide methods of construction of the described machine-learning framework. Following this, methods of use of the MAIVeSS framework for identifying vaccine seed virus candidates will be provided.

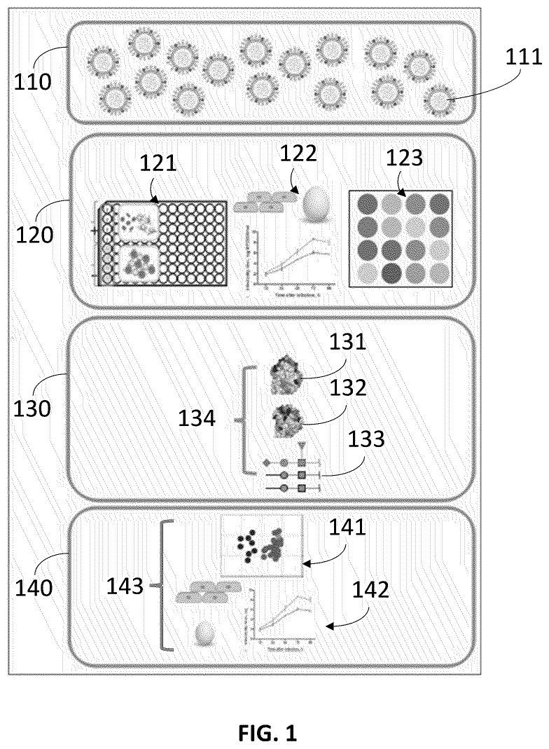

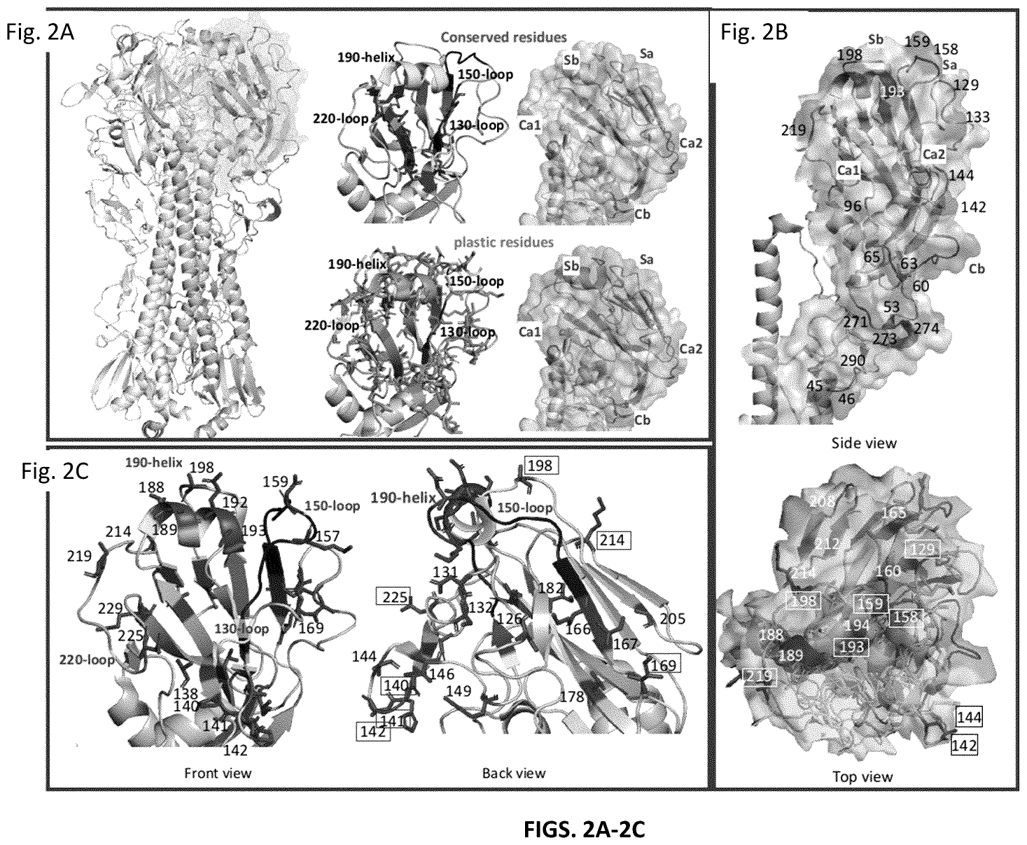

FIG. 1 provides a general outline for the creation of the machine-learning framework, MAIVeSS. The general four step outline of MAIVeSS construction starts with a first step called a mutant library step 110, proceeds to a second step called a phenotyping step 120, then to a third step called a feature selection step 130, and finally to a fourth step called a predictive modeling step 140. In the mutant library step 110, a library of randomly mutated viral genetic material, or mutant library 111, is acquired. The mutant library 111 is a collection of genetic materials that contain deliberately introduced mutations in particular regions of a virus's genes. In various exemplary embodiments, the genetic materials are plasmids that contain versions of the influenza virus HA gene. FIGS. 2A-2C show exemplary modeling data for the protein structure of HA, which is used as a template to develop the mutant library 111 by identifying regions in the HA receptor site where viable mutant viruses can be obtained.

The exemplary mutant library 111 comprises 822 plasmids with each plasmid carrying between one and seven random mutations within or near the HA receptor binding site (RBS). However, one of ordinary skill can envision a different size of the mutant library 111 as well as a different number of random mutations without deviating from the principle of the described MAIVeSS framework. Thus, in various exemplary embodiments, the mutant library 111 can comprise more than 100, 200, 300, 400, 500, 600, 700, 800, 900, 1000, 1500, 2000, 2500, 3000, 3500, 4000, 4500, 5000, or 10,000 genetic materials such as plasmids, and each genetic material can carry 1, 2, 3, 4, 5, 6, 7, 8, 9, 10, 15, 20, 25, 30, 40, 50, 60, 70, 80, 90, 100 or more mutations within or near the HA RBS.

In the phenotyping step 120, the mutant library 111 is subjected to desired phenotype analyses such as antigenic analyses 121, yield analyses 122, and glycan profiling 123. The exemplary antigenic analyses 121 provide information on the extent of antigenic relationships between genetic materials in the mutant library 111 as well as the level of antigenic drift. The antigenic analyses 121 first require that viruses are generated from corresponding genetic material in the mutant library 111. A total of 189 corresponding mutant viruses are generated from the 822 plasmids in the mutant library 111 via reverse genetics. After the viruses are sequenced, the antigenic analyses 121 of these viruses are conducted by performing hemagglutination inhibition (HAI) assays using ferret antisera. By comparing antigenic relationships of the viruses generated from the mutant library 111 against known wild influenza strains, for example, one can identify which viruses have the most closely matched antigenicity to the wild influenza strains. One of ordinary skill in the art can envision variations of the antigenic analyses 121 that are considered to be within the scope of the present description. Thus, in various exemplary embodiments, one could generate up to 10 viruses, more than 10, 20, 30, 40, 50, 60, 70, 80, 90, 100, 120, 150, 200, 300, 400 or more viruses. In various exemplary embodiments, the antigenic analyses 121 could make use of other animal antisera and/or assays that target other proteins relevant to antigenicity, such as neuraminidase.

The exemplary yield analyses 122 are performed in order to determine how substitutions present in viruses generated from the mutant library 111 affect the yields of those viruses in both cells and eggs when compared to parent wild type virus. The yield analyses 122 provide information on ideal vaccine seed virus candidates from the perspective of ease of production, with high-yield production in both cells and eggs being ideal.

The exemplary glycan profiling 123 is performed in order to acquire more data potentially relevant to viral yield, which can correlate with glycan substructure binding properties. The glycan profiling 123 is thus conducted by analyzing the receptor binding properties of the mutant viruses using glycan microarrays comprising 75 glycoforms. This is followed by a matrix of 28 glycan substructure features to group glycans based on their internal and terminal substructures. The glycan profiling 123 also comprises biolayer interferometry assays (BLI) which are used for determining virus receptor binding affinities. Clearly, the above example of the glycan profiling 123 cites specific values that one of ordinary skill in the art will recognize are significantly alterable without departing from the principle of the present description. Thus, in various exemplary embodiments, one can perform glycan profiling using up to 10, more than 20, 30, 40, 50, 60, 70, 80, 90, 100, 150, 200, 300, 500, 1000, or more glycoforms, and by studying 10, more than 20, 30, 40, 50, 60, 70, 80, 90, 100, 150, 200, 300, 500, 1000, or more glycan substructure features. Additionally, this description contemplates that the glycan profiling 123 could utilize other forms of analysis including, for example, instrumental analyses such as surface plasmon resonance (SPR), Biolayer Interferometry (BLI) and computational methods such as molecular dynamics simulations.

Once the phenotyping step 120 is complete and data related to the desired phenotypes such as antigenicity, yield, and glycan binding properties of mutant viruses generated from the mutant library 111 have been collected, the feature selection step 130 takes place. The feature selection step 130 considers which features are to be mathematically integrated into a MAIVeSS predictive model. Thus, the feature selection step 130 comprises selection of exemplary antigenicity features 132, yield features 133, and receptor binding features 134, all of which are selected by a sparse learning model 131. The sparse learning model 131 enables identification, from all collected genetic data, of the key genetic features that contribute to the phenotypes observed in the phenotyping step 120. Briefly, the sparse learning model 131 uses a linear regression loss function with regularization, which permits determination of the most relevant genetic features associated with a given phenotype. The sparse learning model 131 takes into account genetic distance matrices among proteins or glycan sequences, phenotypic differences, and sample numbers. Specific embodiments of the sparse learning model 131 used in selecting antigenicity features 132, yield features 133 and receptor binding features 134 are described in detail in the Examples below.

The exemplary antigenicity features 131 comprise two sets of features, which are amino acid mutations and N-glycosylation sites. In various exemplary embodiments, these two sets of features are further narrowed so that the antigenicity features 131 specifically comprise residues predicted to be on the surface of HA.

The exemplary yield features 132 are selected to identify mutations associated with virus yields in cells as well as in eggs. The yield features 132 utilize two sets of features: amino acid substitutions and N-glycosylation sites. The sparse learning model 131, in various embodiments, is also used to identify synergistic amino acid substitutions associated with virus yield in eggs or cells.

The exemplary receptor binding features 134 are selected with the goal of determining the substructures associated with yield traits in cells and eggs. The receptor binding features 134 comprise terminal substructures, internal substructures, and base substructures which are linked to the glycan microarray.

Once the sparse learning model 131 has been used for feature selection, a predictive model 141 is generated. Essentially, the features selected via the sparse learning model form the basis for the predictive model 141. The predictive model 141 is developed to estimate the antigenic distance between two viruses based on their genetic sequences. Thus, in various exemplary embodiments, HA protein sequences can be used as input data to the predictive model 141. Output from the predictive model 141 includes quantified antigenic distance data 142 and yield difference data 143. Output from the predictive model 141 is calculated on the basis of a genetic distance vector between two viruses, a predicted antigenic distance between the two viruses, a global weight representing an average of weights across different tasks, weights from each individual task, and a weighting parameter p.

The predictive model 141, when used to predict antigenic distance data 142, is generally defined according to Equation 1 below:

$$\hat{y} = x ( μ {w}^{global} + ( 1 - μ ) {w}^{local} ) \qquad Equation 1$$

Output antigenic distance data 142 from the predictive model 141 as calculated according to Equation 1 considers a genetic distance vector between two viruses (x), a predicted antigenic distance between the two viruses (ŷ), a global weight representing an average of weights across different tasks (w^{global}), weights from each individual task(W^{local}), and a weighting parameter p.

The predictive model 141, when used to predict yield difference data 143, also incorporates a scoring function that is generally defined according to Equation 2 below:

$$\hat{y} = {\sum }_{k = 1}^{K} {\sum }_{{i}_{1} , … , {i}_{k}}^{d} {w}_{< {i}_{1 , … , {i}_{k} >}}^{( k )} {z}_{< {i}_{1 , … , {i}_{k} >}}^{( k )} \qquad Equation 2$$

Output yield difference data 143 from the predictive model 141 as calculated incorporating Equation 2 considers w and z as weight and feature matrices, respectively, from the sparse learning model 131. Specific examples of the use and incorporation of Equation 2 into the predictive model 141 are described in detail in the Examples below.

The MAIVeSS method can be used to predict preferred viral strains for vaccine development by focusing analysis on key amino acid residues it has identified to be related to antigenicity and yield. That is, the antigenicity features 132, yield features 133 and receptor binding features 134 selected by the sparse learning model 131 can be used by the predictive model 141 to predict preferred viral strains via output quantified antigenic distance data 142 and yield difference data 143.

Hence, in various exemplary embodiments, one can use the amino acid residues associated with antigenicity in Table 1 below to assess antigenicity of vaccine candidate viruses and thus predict which viruses will have acceptable or optimal antigen matching with the most prevalent wild strains of a virus. In Table 1, the following abbreviations are used to further describe residues: antibody binding site (ABS); receptor binding site (RBS); N-linked glycosylation (Gly); Calcium binding site 1 (Ca1); Calcium binding site 2 (Ca2); Site A (Sa); and Site B (Sb). Bootstrap values shown in Table 1 were derived from 100 independent experiments, each with 80% of the training data.

TABLE 1

| Residue | Bootstrap | w^{global} (±SD) |
|---|---|---|
| 45 | 100 | 0.1019 |
| | | (0.0051) |
| 46 | 93 | 0.0337 |
| | | (0.0018) |
| 53 | 100 | 0.3728 |
| | | (0.0018) |
| 60 | 100 | 0.4071 |
| | | (0.0118) |
| 63 (Gly) | 100 | 0.2220 |
| | | (0.0067) |
| 65 | 100 | 0.0748 |
| | | (0.0035) |
| 96 | 100 | 0.0424 |
| | | (0.0015) |
| 125c | 100 | 0.2380 |
| | | (0.0024) |
| 129 (Gly) | 100 | −0.1397 |
| | | (0.0057) |
| 133 | 89 | 0.5540 |
| | | (0.0325) |
| 133a | 89 | 0.0419 |
| | | (0.0034) |
| 142 (Ca2) | 100 | 0.1033 |
| | | (0.0026) |
| 144 (Ca2) | 100 | 0.1322 |
| | | (0.0037) |
| 158 (Sa, | 100 | 0.2550 |
| Gly) | | (0.0010) |
| 159(Sa) | 100 | 0.3895 |
| | | (0.0131) |
| 160(Sa) | 94 | 0.0312 |
| | | (0.0014) |
| 165(Sa) | 100 | −0.0286 |
| | | (0.0016) |
| 188(Sb) | 90 | 0.1739 |
| | | (0.0105) |
| 189(Sb) | 100 | 0.1138 |
| | | (0.0036) |
| 193(Sb, | 100 | 0.1502 |
| RBS) | | (0.0017) |
| 194 (Sb, | 100 | 0.0918 |
| RBS) | | (0.0017) |
| 198 (Sb) | 100 | 0.0631 |
| | | (0.0007) |
| 208 (Ca1) | 100 | 0.0689 |
| | | (0.0037) |
| 212 | 100 | 0.2921 |
| | | (0.0021) |
| 214 | 88 | 0.0262 |
| | | (0.0013) |
| 219 | 94 | 0.0530 |
| | | (0.0037) |
| 271 | 100 | −0.0244 |
| | | (0.0035) |
| 273 | 100 | 0.0684 |
| | | (0.0021) |
| 274 | 100 | 0.6435 |
| | | (0.0050) |
| 290 | 82 | 0.0649 |
| | | (0.0036) |

Global weights (w^{global}) as listed in Table 1 were learned from an application of the sparse learning model 131 called MTL-GGSL, further detailed in the Examples below, and absolute local weights for each individual task are provided in U.S. provisional patent application Ser. No. 63/578,043, which is hereby incorporated by reference in its entirety.

In various exemplary embodiments, one can use the amino acid residues associated with yield in Table 2 below to predict which viruses will have acceptable or optimal yields for the purpose of vaccine production.

TABLE 2

| | Yield Trait | | | | | | | | | | | |
|---|---|---|---|---|---|---|---|---|---|---|---|---|
| | | Boot- | Weight | | Boot- | Weight | | Boot- | Weight | | Boot- | Weight |
| Residue | HY^{cell} | strap | (±SD) | HY^{egg} | strap | (±SD) | LY^{cell} | strap | (±SD) | LY^{egg} | strap | (±SD) |
| 126 | P to SP | 81 | 0.5705 | P to SP | 80 | 1.6838 | — | | | — | | |
| | | | (0.2311) | | | (0.6509) | | | | | | |
| 131 | — | | | P to P | 100 | 0.2478 | P to P | 99 | −0.2527 | — | | |
| | | | | | | (0.2018) | | | (0.2014) | | | |
| 132 | P to NP | 87 | 0.4785 | P to NP/SP | 85 | 0.7507 | P to SP | 99 | −0.5006 | — | | |
| | | | (0.2082) | | | (0.3073) | | | (0.1974) | | | |
| 133a | P to NP/P | 99 | 0.5869 | P to NP/P | 100 | 1.7985 | — | | | — | | |
| | | | (0.0892) | | | (0.4504) | | | | | | |
| 137 | — | | | — | | | SP to NP/P | 94 | −1.4894 | SP to NP/P | 95 | −0.4997 |
| | | | | | | | | | (0.6504) | | | (0.1827) |
| 138 | SP to P | 82 | 0.0693 | SP to P | 82 | 0.9026 | — | | | — | | |
| | | | (0.1157) | | | (0.3905) | | | | | | |
| 140 (Ca2) | SP to P | 100 | 0.1603 | — | | | SP to P | 95 | −0.3991 | — | | |
| | | | (0.3925) | | | | | | (0.2088) | | | |
| 141 (Ca2) | P to P | 100 | 0.9139 | P to NP/P | 100 | 1.0663 | P to NP | 98 | −0.2150 | — | | |
| | | | (0.4540) | | | (0.3840) | | | (0.7936) | | | |
| 142 (Ca2) | SP to NP/SP | 93 | 0.2102 | SP to NP | 96 | 1.1147 | SP to P | 100 | −0.8497 | SP to P/SP | 96 | −0.1094 |
| | | | (0.4026) | | | (0.3030) | | | (0.2313) | | | (0.2109) |
| 144 (Ca2) | SP to | 81 | 0.3179 | SP to | 82 | 1.1002 | — | | | — | | |
| | NP/SP/P | | (0.1226) | NP/SP/P | | (0.4648) | | | | | | |
| 146 | — | | | P to NP/SP | 99 | 0.4318 | P to | 100 | −1.1819 | P to P | 84 | −0.0683 |
| | | | | | | (0.1459) | P/NP/SP | | (0.4838) | | | (0.0289) |
| 149 | — | | | P to NP/SP | 96 | 0.3256 | P to NP/SP | 99 | −0.5463 | — | | |
| | | | | | | (0.3971) | | | (0.3416) | | | |
| 157 (Sa) | P to NP/P | 100 | 0.5483 | P to NP/P | 100 | 1.1876 | — | | | — | | |
| | | | (0.6302) | | | (0.3793) | | | | | | |
| 159 (Sa) | P to P | 99 | 0.7856 | P to P | 97 | 0.0379 | P to NP | 85 | −0.0439 | P to NP | 88 | −1.2852 |
| | | | (1.1559) | | | (1.1499) | | | (0.1606) | | | (0.6344) |
| 162 (Sa) | — | | | — | | | SP to P | 99 | −0.7152 | SP to P | 100 | −0.1045 |
| | | | | | | | | | (0.3527) | | | (0.5025) |
| 166 (Sa) | P to NP/P | 100 | 0.4145 | P to NP/P | 99 | 1.5899 | — | | | — | | |
| | | | (0.8401) | | | (1.2154) | | | | | | |
| 167 (Sa) | — | | | P to P | 100 | 0.8343 | P to P | 100 | −0.0396 | — | | |
| | | | | | | (0.2141) | | | (0.3050) | | | |
| 169 (Ca1) | NP to NP | 100 | 0.5871 | NP to NP | 100 | 1.8680 | NP to P | 100 | −0.1047 | NP to P | 99 | −0.9964 |
| | | | (0.4362) | | | (0.5976) | | | (0.3531) | | | (0.1964) |
| 173(Ca1) | — | | | — | | | SP to P | 100 | −0.3385 | SP to P | 100 | −0.9965 |
| | | | | | | | | | (0.2147) | | | (0.3552) |
| 174 | — | | | — | | | P to P | 99 | −0.5564 | P to P | 99 | −0.1380 |
| | | | | | | | | | (0.2064) | | | (0.6943) |
| 178 | — | | | NP to NP | 99 | 0.1548 | NP to P | 85 | −1.1321 | — | | |
| | | | | | | (0.0990) | | | (0.2201) | | | |
| 182 | NP to NP | 85 | 1.2553 | NP to NP | 84 | 2.4218 | — | | | — | | |
| | | | (0.5159) | | | (1.0087) | | | | | | |
| 188 (Sb) | P to SP/P | 83 | 0.8182 | P to SP | 89 | 0.1816 | P to NP | 83 | −0.1794 | P to NP/P | 88 | −1.2478 |
| | | | (0.3406) | | | (0.0669) | | | (0.2453) | | | (0.5429) |
| 189 (Sb) | SP to NP | 100 | 0.2487 | SP to P | 100 | 0.7942 | SP to P | 99 | −0.2922 | SP to NP | 100 | −0.0306 |
| | | | (0.4659) | | | (0.1624) | | | (0.2709) | | | (0.0891) |
| 192 (Sb) | — | | | P to NP | 98 | 0.3159 | P to NP | 93 | −0.2301 | — | | |
| | | | | | | (0.2751) | | | (0.1982) | | | |
| 193 (Sb, | P to P | 88 | 1.8718 | P to P/NP | 82 | 2.5413 | P to SP/NP | 82 | −1.1719 | P to SP | 86 | −0.3178 |
| RBS) | | | (0.9340) | | | (1.1365) | | | (0.4636) | | | (0.1401) |
| 197 (Sb) | — | | | — | | | P to P | 97 | −0.0369 | P to P | 93 | −0.2856 |
| | | | | | | | | | (0.6627) | | | (0.4440) |
| 198 (Sb) | — | | | SP to NP/P | 100 | 1.1147 | SP to | 100 | −0.6787 | SP to SP | 81 | −0.2478 |
| | | | | | | (0.9888) | SP/NP/P | | (0.3430) | | | (0.2074) |
| 205 | SP to SP | 82 | 0.9085 | SP to SP | 80 | 1.2537 | SP to P | 98 | −0.1013 | SP to P | 100 | −0.5482 |
| | | | (0.3800) | | | (0.3943) | | | (0.4861) | | | (0.4787) |
| 206 (Ca1) | — | | | — | | | P to P | 99 | −0.2549 | P to P | 100 | −0.6986 |
| | | | | | | | | | (0.7150) | | | (0.7722) |
| 211 | — | | | — | | | P to P | 100 | −0.5541 | P to P | 99 | −0.6789 |
| | | | | | | | | | (0.2891) | | | (0.3159) |
| 212 | — | | | — | | | P to NP/P | 97 | −0.4308 | P to NP/P | 100 | −0.7254 |
| | | | | | | | | | (0.1808) | | | (0.2640) |
| 214 | P to NP/P | 100 | 0.3179 | P to NP/P | 100 | 1.5003 | — | | | — | | |
| | | | (0.1253) | | | (0.5523) | | | | | | |
| 219 | — | | | NP to P | 95 | 0.2334 | NP to NP/P | 100 | −0.8865 | NP to NP | 98 | −0.0765 |
| | | | | | | (1.0338) | | | (0.1492) | | | (0.1014) |
| 222 (RBS) | — | | | — | | | P to P | 100 | −0.6312 | P to P | 99 | −0.6276 |
| | | | | | | | | | (0.2283) | | | (0.3037) |
| 225 (Ca2, | P to SP | 100 | 0.2013 | P to P | 96 | 0.8162 | P to P | 96 | −0.1763 | P to SP | 99 | −0.5636 |
| RBS) | | | (0.2212) | | | (0.3808) | | | (0.1970) | | | (0.2010) |
| 229 | P to P | 97 | 0.0123 | — | | | — | | | P to P | 93 | −0.0520 |
| | | | (0.1321) | | | | | | | | | (0.2880) |
| 237 | — | | | — | | | NP to NP | 100 | −0.1318 | NP to NP | 100 | −0.6804 |
| | | | | | | | | | (0.2421) | | | (0.2477) |

Table 2 uses the following abbreviations for amino acids: nonpolar (NP), which includes amino acids valine (V), leucine (L), isoleucine (1), methionine (M), cysteine (C), phenylalanine (F), tryptophan (W) and tyrosine (Y); small polar (SP), which includes amino acids glycine (G), alanine (A) and proline (P); and polar/charged (P), which includes amino acids serine (8), threonine (T), asparagine (N), glutamine (0), histidine (H), aspartic acid (D), glutamic acid (E), lysine (K) and arginine (R). Substitutions listed in Table 2 that are associated with HY^{both} or LY^{both} are bolded. Cells in Table 2 with ‘—’ denotes that it is unknown how the corresponding residue will affect yield.

### Examples

The following examples comprise descriptions of exemplary embodiments of the herein disclosed method of analysis. These examples are not intended to be limiting or to define the scope of the present disclosure.

### Machine-Learning Assisted Influenza Vaccine Strain Selection Framework (MAIVeSS).

Machine learning models have been shown to be effective in identifying antigenicity associated features in protein sequences from different subtypes of influenza A viruses. Machine learning models were developed to identify the specific sequence features in HA proteins that determine three important phenotypes: antigenicity, yield in cells and eggs, and receptor binding. To achieve this, the models were trained on large datasets of HA protein sequences and associated phenotype information. A quantitative function was developed that allows us to measure the distances between sequences based on their phenotypic characteristics. Ultimate goals for these machine learning models are to identify: 1) mutations in the HA RBS that affect virus antigenicity; 2) mutations in the HA RBS that increase or decrease virus yields in cells and/or eggs; and 3) specific glycan substructures (glycan motifs) on the surface of cells or eggs that are associated with increased yields of influenza virus. By achieving these goals, the hope is to gain a better understanding of the molecular determinants of these important viral phenotypes and to identify potential targets for the development of improved influenza vaccines.

The problem of identifying genetic features associated with influenza virus phenotypes was approached using a sparse learning model. Mathematically, this model involves a linear regression loss function with regularization, which allows us to determine the most relevant genetic features associated with a given phenotype. The sparse learning model combines a least squares loss with a regularized term and takes into account genetic distance matrices among HA proteins or glycan sequences (denoted as X), phenotypic differences (denoted as y), and sample numbers (denoted as N). This approach enables us to identify the key genetic features that contribute to different influenza virus phenotypes, such as antigenicity, yield, and receptor-binding.

The objective of the sparse learning model is to solve: min L(X, y, w)+λR(w) where L(X,y,w) is the loss function, A is a pre-defined regularization parameter, R(w) denotes the regularization term, and denotes the numerical weights of individual features (either a single residue or a group of neighboring residues). Absolute values of the weights indicate the impact of each mutation of a specific feature to phenotypes (i.e., antigenic, yield, and receptor-binding properties). The larger the absolute weight, the greater the impact.

Based on the features learned from sparse learning, a predictive model was developed to assess antigenic or yield properties given HA sequences. Specifically, ŷ=xw, where ŷ is the predicted phenotypic distance (either antigenicity or yield) between the two viruses; x is the feature distance vector; and w is the weight vector for those features, which can be associated with either antigenicity or yield.

Multi-task learning group-guided sparse learning (MTL-GGSL) model. To address the challenges associated with integrating serological data generated from different platforms (e.g., turkey and guinea red blood cells), a Multi-Task Learning (MTL) approach was utilized with Group Graphical Sparse Learning (GGSL) to analyze antigenicity. This approach allowed us to consider both N-linked glycosylation and amino acid features when analyzing the data. MTL allows us to learn multiple related tasks (i.e., analyzing antigenicity from different serological platforms) simultaneously, while GGSL considers the dependencies between different groups of features to improve the accuracy of the analysis. By utilizing MTL-GGSL, one is able to overcome the challenges associated with integrating data from different platforms and provide a more comprehensive analysis of antigenicity.

One advantage of using the group LASSO regularization in MTL-GGSL for antigenicity analyses is that it encourages multiple predictors from related tasks to share a subset of features. This is in contrast to the LASSO regularization, which may lead to sparse solutions where only a few features are selected for each task independently. Previous study has shown that incorporating information on N-linked glycosylation can improve the performance of sparse learning models in predicting antigenic properties of influenza viruses.

By adopting MTL-GGSL, one is able to integrate information on both glycosylation and amino acid sequences from serological data generated using different platforms, which can further enhance the accuracy of the predictive models described herein for influenza antigenicity.

Specifically, the following was defined:

$$L ( X , y , w ) = \frac{1}{2} {\vert \vert Y - XW \vert \vert}_{F}^{2} ,$$

$$λ R ( W ) = {λ}_{1} {R}_{1} ( W ) + {λ}_{2} {R}_{2} ( W ) + {λ}_{3} {R}_{3} ( W ) ,$$

and the model is formulated as:

$${min}_{w} \frac{1}{2} {\vert \vert Y - XW \vert \vert}_{F}^{2} + {λ}_{1} {\sum }_{j = 1}^{p} {\vert \vert {W}_{j .} \vert \vert}_{1} + {λ}_{2} {\sum }_{t = 1}^{k} {\sum }_{l = 1}^{q} {α}_{l} {\vert \vert {W}_{{G}_{l} , t} \vert \vert}_{2} + {λ}_{3} {\sum }_{t = 1}^{k} {\sum }_{l = 1}^{q} {α}_{l} {\vert \vert {W}_{{G}_{l} , t} \vert \vert}_{1} ,$$

where λ₁, λ₂, and λ₃ are regularization parameters, j is the subscript for feature, p is the total number of features, Gₗ denotes the feature group, q is the number of feature groups, αₗ=\sqrt{mₗ} is the weight of feature group Gₗ; Wj denotes the weights for the j-th feature among different tasks, and W_{Gl,t} is the weight for feature group Gₗ of the t-th task. Alternating Direction Method of Multipliers (ADMM) was employed to solve the optimization problem.

### The Generalized Hierarchical Sparse Model (GHSM)

To consider synergistic effects of multiple features on the phenotypes, GHSM was adopted. The GHSM model aims to minimize:

$$L ( W ) + {\sum }_{k = 1}^{K} \frac{λ}{{a}^{k}} {\vert \vert {W}^{( k )} \vert \vert}_{1} .$$

GHSM model solves the following objective:

$${min}_{w} \frac{1}{2} {‖ y - {\sum }_{k = 1}^{K} {\sum }_{{i}_{1 , … ,} {i}_{k}}^{d} {w}_{< {i}_{1 , … ,} {i}_{k} >}^{( k )} {z}_{< {i}_{1 , … ,} {i}_{k} >}^{( k )} ‖}^{2} + {\sum }_{k = 1}^{K} \frac{λ}{{α}^{k}} {‖ {w}^{( k )} ‖}_{1}$$

$$s . t . \vert {w}_{i}^{( 1 )} \vert \geq {‖ {e}_{i}^{( 2 )} ⊙ {w}^{( 2 )} ‖}_{1} \geq … \geq {‖ {e}_{i}^{( K )} ⊙ {w}^{( K )} ‖}_{1} , i ∈ {ℕ}_{d}$$

Above, λ and α are two regularization parameters controlling the sparsity and the decay in the coefficients for interactions of different orders, Z_{<i}_{₁}_{, . . . , i}_{ₖ}_{>}^{(k)} denotes a data vector for the k-th order interaction corresponding to <i₁, . . . , iₖ>, an interaction index <iᵢ, . . . , iₖ>, where i₁< . . . <iₖ is an index to uniquely indicate the interaction among the covariates i₁, . . . , iₖ, W denotes the set of parameters

$${\{ {w}^{( k )} \}}_{k = 1}^{K} , {w}^{( k )} ∈ {ℝ}^{{(}_{k}^{d} )} for k = 1 , … ,$$

K is a vector of length

$${(}_{k}^{d} ) = \frac{d !}{k ! ( d - k ) !}$$

with w_{<i}_{₁}_{, . . . , i}_{ₖ}_{>}^{(k)} as its element corresponding to the index <i₁, . . . , iₖ>, ∥·∥₂ denotes l₂ norm of a vector and Q denotes the element wise product of two vectors. The constraints associated with each covariate I have a chain of (K-1) inequality constraints, and there is a total of d chains. The application of those models for antigenicity, yield, receptor-binding are detailed as below.

### Antigenicity Analyses

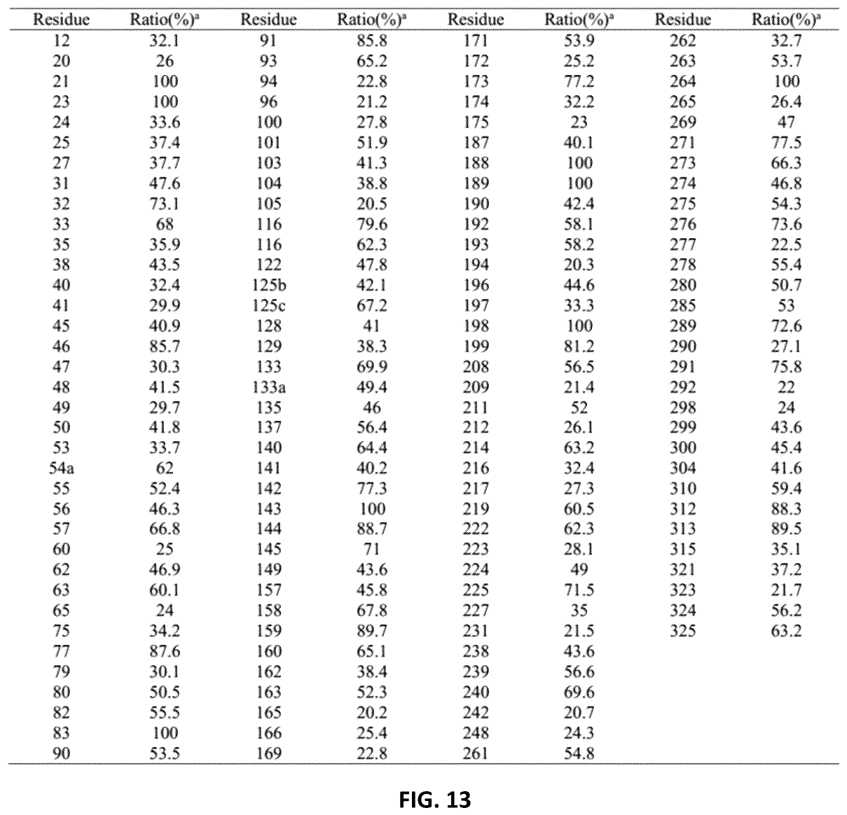

In this Example, eight individual tasks were used, each corresponding to an individual HAI dataset, including those for seasonal H1N1 viruses (1977-2009), 2009 H1 N1 viruses (2009-2020), swine H1 N1 viruses, and mutants generated above. In each task, the low-rank matrix completion algorithm was used to minimize data noise and the challenges derived from low reactors and missing values in the HAI datasets, and antigenic cartography was then used for antigenic distance calculation. Two groups of features (i.e., amino acid mutations and N-glycosylation sites) were used in the model to quantify influenza antigenic distances. 327 residue features and 6 N-glycosylation site features were defined. GETAREA software was used to predict whether or not residues were on HA's surface. The A(H1N1)pdm09 three-dimensional HA structure (Protein Data Bank [PDB] identifier [ID]3LZG) was used as the template. A total of 138 residues were predicted to be located at the HA protein's surface (FIG. 13). All amino acid residues, with a variant rate >10%, will be considered as non-conserved sites and included in the machine learning model. Finally, a total of 86 residues with 4 N-glycosylation sites were used as features in the machine learning model.

### Yield Analyses.

In this Example, the yield of 189 mutants was analyzed compared with the parent wild type virus in both cells and eggs. To analyze the data, two groups of features were utilized: amino acid substitutions and N-glycosylation sites. Furthermore, the GHSM approach was employed to identify synergistic amino acid substitutions associated with virus yield in eggs or cells.

### Glycan Binding Analyses.

In this Example, a glycan microarray with 75 glycoforms was used, which were grouped based on their internal and terminal substructures and linkers into a matrix of 28 glycan substructure features. The Multi-Task Learning with Group Graphical Sparse Learning (MTL-GGSL) approach was then used to determine the substructures associated with yield traits in cells and eggs. In the model, three groups of features were employed, including terminal substructures (n=17), internal substructures (n=8), and base substructures linked to the array (n=3).

### Model Comparison, Parameter Optimization, and Bootstrapping Analyses

In order to ensure the robustness of these analyses, these results were compared with three other sparse models: the L1-norm regularized method (LASSO),the L2-norm regularized method (RIDGE), and the sparse group LASSO method (SGL). Results were also compared with two additional methods that incorporate both L1- and L2-norm regularization, including the L1- and L2-norm regularized method41 and the L1- and L∞-norm Composite Absolute Penalties method (iCAP).

To investigate the effect of amino acid substitutions on both yield and glycan binding phenotype, a grouping method for amino acids was employed. Each amino acid was assigned to one of three groups based on its biophysical properties: nonpolar (V, L, I, M, C, F, W, and Y), small polar (G, A, and P), and polar/charged (S, T, N, Q, H, D, E, K, and R). HA protein sequence was encoded into a vector by comparing to a wild-type sequence and if a mutation occurred in residue j (e.g., nonpolar to small polar), the j-th element of was encoded to 1; otherwise, it was encoded to 0. To evaluate the directionality of amino acid substitutions on both yield and glycan binding phenotype, three different sparse models (LASSO, RIDGE, and SGL) were used and parameter optimization and bootstrap analyses were performed. In brief, all features with A bootstrap value cutoff of 80 from 100 independent runs were selected.

### Predictive Model

In this Example, a predictive model was developed to estimate the antigenic distance between two viruses based on their genetic sequences. The model was defined as follows: ŷ=x(μw^{global}+(1−μ)w^{local}), where x is the genetic distance vector between the two viruses, ŷ is the predicted antigenic distance between them, W^{global} is the global weight representing the average of weights across different tasks, w^{local} indicates the weights from each individual task, and μ is set to 0.4 to balance the global and local weights.

In addition, a scoring function was proposed to measure yield differences between two viruses based on their amino 398 acid sequences. The scoring function is defined as follows:

$$\hat{y} = {\sum }_{k = 1}^{K} {\sum }_{{i}_{1} , … , {i}_{k}}^{d} {w}_{< {i}_{1 , … , {i}_{k} >}}^{( k )} {z}_{< {i}_{1 , … , {i}_{k} >}}^{( k )}$$

Here, w and z were the weight and feature matrices used in the GHSM approach mentioned above. The detailed prediction results for both the antigenic distance and yield differences are presented in U.S. provisional patent application Ser. No. 63/578,043, referenced herein.

### Cells and Viruses.

405 The human embryonic kidney (293T) cells and Madin-Darby canine kidney (MDCK) CCL-34 cells were obtained 406 from the American Type Culture Collection (Manassas, VA). The cells were maintained in Dulbecco's Modified 407 Eagle Medium (GIBCO/BRL, Grand Island, NY) supplemented with 5% fetal bovine serum (Atlanta Biologicals, 408 Lawrenceville, GA) and penicillin-streptomycin (Invitrogen, Carlsbad, CA) at 37° C. with 5% C02. The HA gene 409 of CA/04 was cloned into the vector pHW2000 and used as a template to construct the mutant library. The viruses 410 generated by reverse genetics were propagated in MDCK cells and cultured at 37° C. with 5% C02 in Opti-MEM 411 medium (GIBCO/BRL, Grand Island, NY) supplemented with 1 μg/ml of TPCK (N-tosyl-L-phenylalanine 412 chloromethyl ketone)-Trypsin (Sigma-Aldrich, St. Louis, MO) and penicillin-streptomycin (Invitrogen, Carlsbad, 413 CA). The virus titers were determined by TCID50 in MDCK cells.

### Sequence and Serological Data.

Serologic data for H1 N1 viruses were collected from data described elsewhere including 2,030 HAI titers generated between 153 viruses and 97 serum samples. A total of 3,080 non-identical 2009 H1 N1 protein sequences from 2019 to 2020 were obtained from GISAID (https://gisaid.org).

### Construction of Plasmid Library, Gene Synthesis, and Rescue of Mutants.

The mutant plasmid library with random mutations in the HA RBS was generated using the epPCR strategy, as previously described. Four primers were used to generate the HA-pHW2000 RBS mutant library: 1) 130loop_F: 5′-TCA TGG CCC AAT CAT GAC TCG AAC-3′ (SEQ ID NO:1); 2) 190helix_F: 5′-TGG GGC ATT CAC CAT CCA TCT ACT-3′ (SEQ ID NO:2); 3) 190helix_R: 5′-AAC ATA TGT ATC TGC ATT CTG ATA-3′ (SEQ ID NO:3); and 4) 220loop_R: 5′-TAG TGT CCA GTA ATA GTT CAT TCT-3′ (SEQ ID NO:4). The epPCR product (2 μl) was transfected into XL1-Blue Supercompetent Cells (Agilent Technologies, Santa Clara, CA). The transformed cells were directly inoculated onto LB (Luria Bertani) agar plates, and the clones were propagated in 5 ml of LB media. The clones generated from the RBS mutant library were confirmed by Sanger sequencing using the sequencing primer 5′-GAA CGT GTT ACC CAG GAG ATT-3′ (SEQ ID NO:5). Mutant viruses were rescued by plasmid-based reverse genetics with the NA genes from CA/04 and six internal genes from PR8, as described elsewhere.

To compare the phenotypes of the predicted vaccine candidates, a wild-type reassortant virus (rg-WT) was also generated with wild-type HA and NA genes from CA/04 and six internal genes from PR8 using reverse genetics.

To validate the antigenic and high-yield properties of the viruses predicted by the computational model described herein, the HA and NA genes for four potential vaccine candidates were synthesized from epidemic strains (Gene Universal Inc., Newark, DE) and then generated reassortant viruses with the HA and NA from each of these testing epidemic strains and the six internal genes from PR8 using reverse genetics: A/Saint-Petersburg/R1157/2016(H1 N1) (HA,NA)×PR8(rgSP/16), A/Chongqing-Yuzhong/SWL1453/2017(H1 N1)(HA,NA)×PR8(rgCQ/17), A/Brunei/25/2019(H1 N1)(HA,NA)×PR8(rgBRU/19), and A/Malaysia/33075487/2020(H1 N1)(HA,NA)×PR8(rgMAS/20).

### Evaluation of Viral Yield.

To evaluate the effect of mutations on viral yield, cell culture assays and embryonated egg assays were performed. For the cell culture assays, MDCK cells were inoculated with each influenza virus at a multiplicity of infection of 0.001 TCID50 and incubated the cells at 37° C. with 5% C02 for 1 hour. After incubation, the inocula were removed, and the cells were washed twice with phosphate-buffered saline (PBS). Then, the cells were incubated with Opti-447 MEM I (GIBCO, Grand Island, NY) containing TPCK-trypsin (1 μg/ml) at 37° C. with 5% C02. After 48 hours, 200 μl of supernatants were collected, aliquoted, and stored at −80° C. until use. For the embryonated egg assays, 9-day-old specific pathogen-free chicken eggs were inoculated with 200 TCID50 of each virus and incubated at 37° C. for 72 hours, and allantoic fluid were collected. The viral titers in the samples from both the MDCK cells and the embryonated eggs were determined using TCID50 assays in MDCK cells.

### Virus Concentration and Purification.

Viruses for the glycan microarray analysis were purified as previously described. Briefly, viruses were purified from the cell supernatant or allantoic fluid by low-speed clarification (2,482×g, 30 min, 4° C.) to remove debris and then followed by ultracentrifugation through a cushion of 30%-60% sucrose in a 70 Ti Rotor (Beckman Coulter, Fullerton, CA) (100,000×g, 3 h, 4° C.). The virus pellet was re-suspended in 100 μl of PBS and stored at −80° C. until use.

### Glycan Microarray

To identify unique substructures bound specific sets of mutants, a glycan microarray with 75 glycoforms were printed on N-hydroxysuccinimide (NHS)-derivatized slides as described previously.

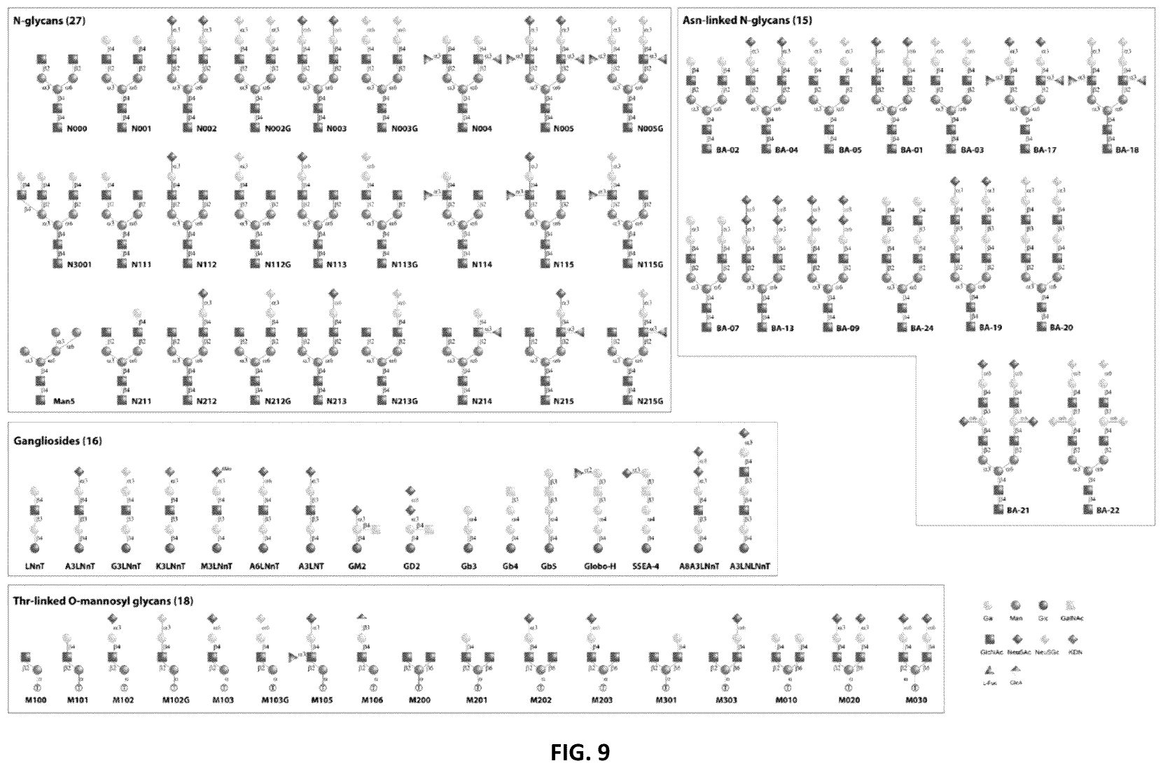

The 75 glycans were selected to represent four different glycan categories, including N-glycans, Asn-linked N-glycans, Gangliosides, Thr-linked O-mannosyl glycans (FIG. 9). These glycans on the microarray have the same base structures and spacer arms but different terminal structures. The glycans were printed in replicates of four in a subarray, and sixteen subarrays were printed on each glass slide. All glycans were prepared at a concentration of 100 mM in phosphate buffer (100 mM sodium phosphate buffer, pH 8.5). The slides were fitted with a 16-chamber adapter to separate the subarrays into individual wells for assay. The unreacted NHS groups on the slides are blocked with 50 mM ethanolamine in 50 mM sodium borate buffer (pH 9.2) at 4° C. for 1 hour, and then, the slides are rinsed with water. Before the assay, slides were rehydrated for 5 min in TSMW buffer (20 mM Tris-HCl, 150 mM NaCl, 0.2 mM CaCl₂), and 0.2 mM MgCl₂, 0.05% Tween). Viruses are purified by sucrose density gradient ultracentrifugation and titrated to about 32,000 hemagglutination units/mi. Then 10 μl of 1.0 M sodium bicarbonate (pH 9.0) was added to 80 μl of virus, and the virus was incubated with 10 μg of Alexa Fluor 488 NHS Ester (Succinimidyl Ester, Invitrogen, Carlsbad, CA) for 1 h at 25° C. After overnight dialysis to remove excess Alexa 488, viruses HA titer were checked and then bound to glycan array. Labeled viruses were incubated on the slide at 4° C. for 2 h, washed, and centrifuged briefly before 476 being scanned with an InnoScan 1100 AL fluorescence imager (Innopsys, Carbonne, France).

Haemagglutination and HAI assays.

Haemagglutination and HAI assays were performed by using 0.5% turkey erythrocytes as described by the WHO Global Influenza Surveillance Network Manual for the Laboratory Diagnosis and Virological Surveillance of Influenza. Turkey erythrocytes were obtained from Lampire Biological Products (Everett, PA). The turkey erythrocytes were washed three times with 1×PBS (pH 7.2) before use and then diluted to 0.5% in 1×PBS (pH 483 7.2).

### Biolayer Interferometry Assays (BLI).

The virus receptor binding affinities were determined by BLI with an Octet RED instrument (Pall ForteBio, Menlo 487 Park, CA). Five biotinylated glycan analogs, Neu5Acα2-3Galβ1-4GlcNacβ-PAA-biotin (3′SLN), Neu5Acα2-488 6Galβ1-4GlcNacβ-PAA-biotin (6′SLN) (Lectinity Holdings, Moscow, Russia), Neu5Acα2-3Galβ1-4(Fucβ1-3)GlcNacβ-PAA-biotin (sLeX), Neu5Gca2-3Galβ1-4GlcNAcβ-PAA-biotin (3′SLN(Gc)), or Neu5Gcα2-3Galβ1-4(Fucβ1-3]GlcNAcβ-PAA-biotin (SLeX(Gc))] were used. Among them, SLeX, 3′SLN(Gc), and SLeX(Gc)were synthesized. The glycans were preloaded onto streptavidin-coated biosensors at up to 0.3 μg/ml for 3 minutes in 1×kinetic buffer (Pall FortéBio, Menlo Park, CA). Each test virus was diluted to a final concentration of 100 pM with 1×kinetic buffer containing 10 μM oseltamivir carboxylate (American Radiolabeled Chemicals, St. Louis, MO) and zanamivir (Sigma-Aldrich, St. Louis, MO) to prevent cleavage of the receptor analogs by NA proteins of virus. Association was measured for 30 minutes at 25° C. Responses were normalized by the highest value obtained during the experiment, and binding curves were fitted by using the binding-saturation method in GraphPad Prism 8. The normalized response curves report the fractional saturation (f) of the sensor surface as described in elsewhere. The RSL0.5 values were calculated to determine the binding affinity between a virus and glycan analog pair, using the binding-saturation method in GraphPad Prism 8 software. Higher RSL0.5 values indicate weaker binding affinity between the virus and glycan analog.

### Structural Modeling and Visualization of Proteins Structure.

The three-dimensional structure of HA protein was modeled based on the crystal structure of CA/04 HA in complex with 6′SLN (PDB ID #3UBN) and 3′SLN (PDB ID #3UBQ). Coot was first used to introduce the desired mutation to the three subunits of a HA trimer. The mutated coordinates were subsequently refined by energy minimization using Phenix (https://phenix-online.org). Structure figures were made using Pymol (The PyMOL Molecular Graphics System, Version 1 0.3, Schrödinger, LLC).

### Serological Data

Serological data for exemplary vaccine candidates generated using the methods described herein are provided in Table 3 below. The HAI assays were performed in triplicate using 0.5% turkey red blood cells. The homologous HAI titers are highlighted in bold. Ferret antisera were produced by infecting influenza seronegative ferrets (see details in Supplementary Information) or obtained from BEI Resources or International Reagent Resource.

TABLE 3

| | Ferret Antisera | | | | |
|---|---|---|---|---|---|
| Viruses | CA/04 | CA/07 | UT/09 | MI/15 | WI/19 |
| A/California/04/2009(H1N1pdm) (CA/04) | 320.00 | 640.00 | 320.00 | 320.00 | 40.00 |
| A/California/07/2009(H1N1pdm) (CA/07) | 640.00 | 640.00 | 320.00 | 640.00 | 40.00 |
| A/Utah/20/2009(H1N1)pdm09 (UT/09) | 1280.00 | 160.00 | 1280.00 | 160.00 | 80.00 |
| A/Michigan/45/2015(HA, NA) (MI/15) | 640.00 | 1280.00 | 640.00 | 1280.00 | 80.00 |
| A/Wisconsin/588/2019(H1N1) (WI/19) | 20.00 | 20.00 | 80.00 | 80.00 | 2560.00 |
| A/Saint-Petersburg/RII57/2016 (H1N1)(HA, NA) × PR8 | 640 | 640 | 320 | 640 | 40 |
| (rgSP/16) | | | | | |
| A/Chongqing-Yuzhong/SWL1453/2017(H1N1)(HA, NA) × PR8 | 160 | 80 | 320 | 160 | 40 |
| (rgCQ/17) | | | | | |
| A/Brunei/25/2019(H1N1)(HA, NA) × PR8 (rgBRU/17) | 10.00 | 10.00 | 10.00 | 20.00 | 160.00 |
| A/Malaysia/33075487/2020(H1N1)(HA, NA) × PR8 | 20.00 | 10.00 | 80.00 | 40.00 | 1280.00 |
| (rgMAS/17) | | | | | |

### Machine Learning Assisted Influenza Vaccine Strain Selection Framework (MAIVeSS)

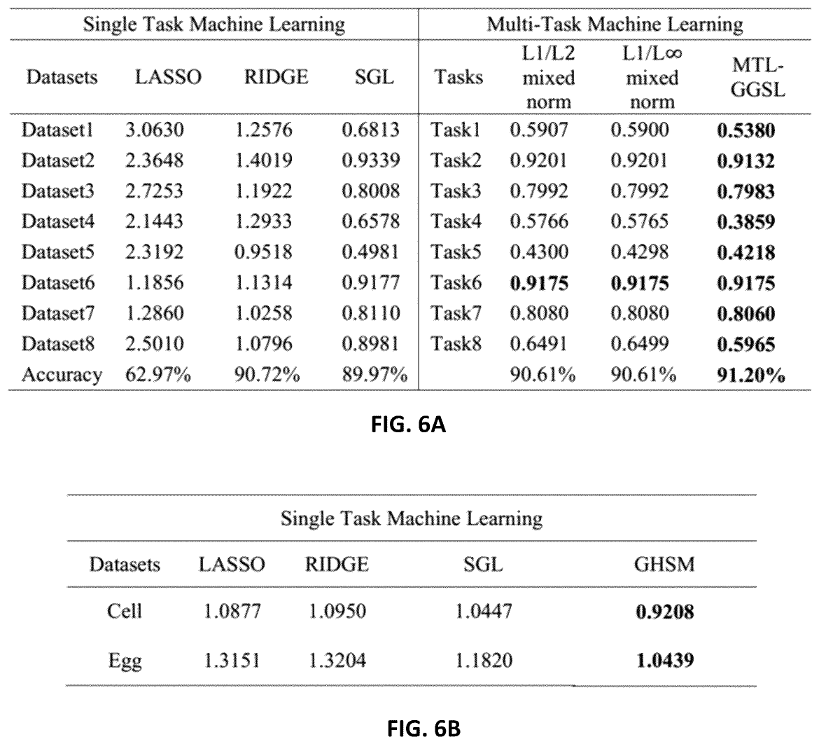

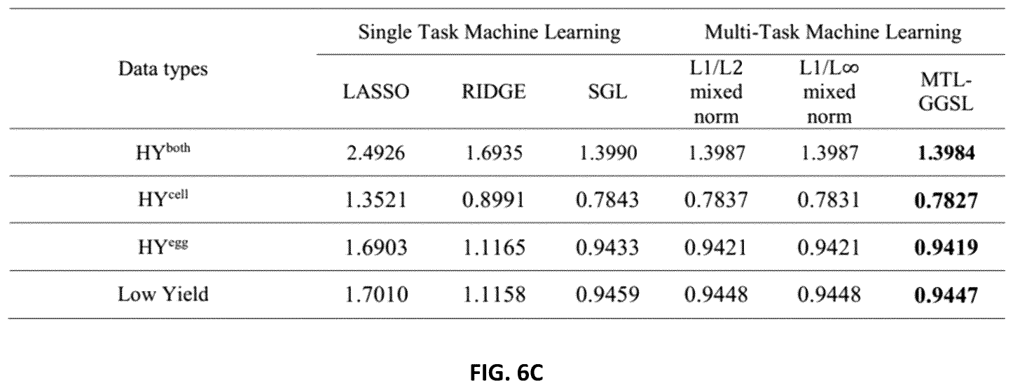

This Example aimed to develop MAIVeSS to learn genetic features associated with three key biological properties for influenza viruses: antigenicity, yield and receptor-binding (FIG. 1). A set of machine-learning models within MAIVeSS were implemented and compared, and it was found that the multi-task learning model (MTL-GGSL) outperformed other state-of-the-art models for assessing antigenicity and glycan binding, while the generalized hierarchical sparse model (GHSM) outperformed other models for assessing yield (see FIGS. 6A-6C).

Using the features learned, MAIVeSS scored vaccine candidates using a query HA protein sequence based on two properties: (1) antigenic properties related to the prototype vaccine antigen, and (2) yield properties in eggs and/or cells (HY^{cell}, high-yield in cells; HY^{egg}, high-yield in eggs; HY^{both}, high-yield in both cells and eggs). High-yield is defined as a >10-fold increase in TCID₅₀/mL compared to the wild-type (WT) in the same substrate. By leveraging these predictive models, MAIVeSS can rapidly identify influenza vaccine candidates that are both antigenically matched and high-yield based on genome sequences obtained during surveillance.

The effectiveness of the machine learning models described herein using A(H1 N1)pdm09 viruses was studied as an exemplary application, but the same principles can be readily applied to other subtypes of influenza viruses.

Development of an a(H1N1)Pdm09 Mutant Library for Machine Learning

To enhance the reliability of feature selection for high-yield viruses, a random mutant virus library that targets the HA receptor binding site (RBS) of A/California/04/2009(H1N1) (CA/04) was established. All the mutants were subjected to antigenic analyses via hemagglutination inhibition (HAI) assays, yield analyses in both MDCK cells and embryonated chicken eggs, and receptor-binding profiling through glycan microarrays. The phenotypic data collected were then used as training and testing data in MAIVeSS to identify the molecular features associated with antigenicity and yield and to establish predictive models.

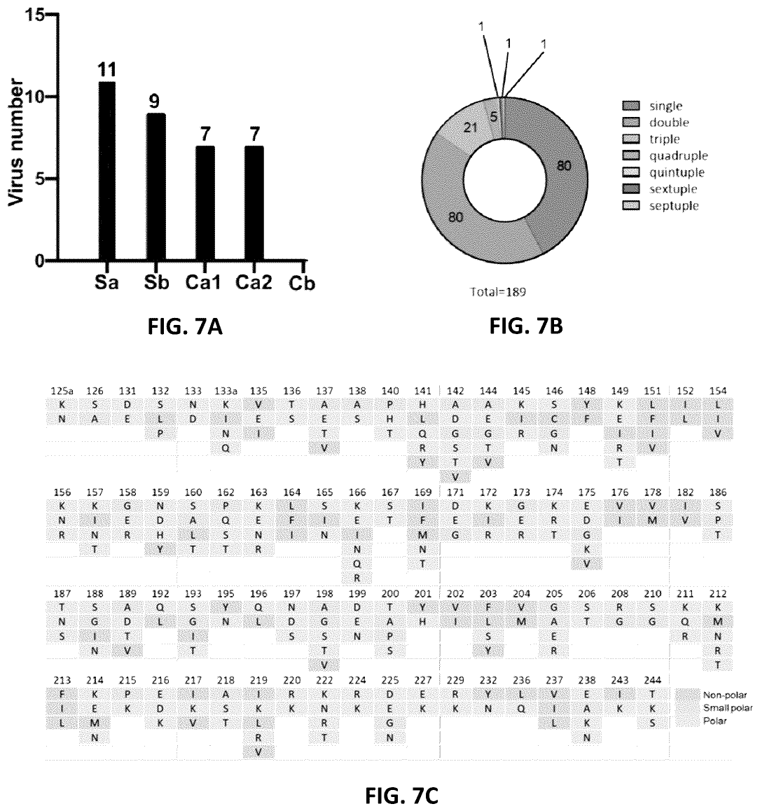

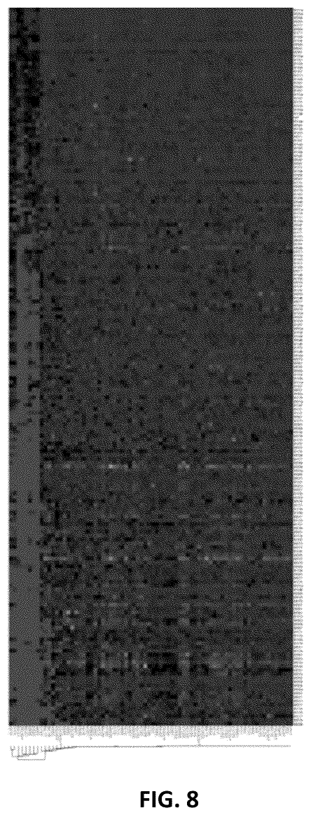

A total of 822 plasmids were generated, each carrying one to seven random mutations within or near the HA RBS (residues 119-241, H1 numbering; 126-244, H3 numbering). Using these mutant plasmids, corresponding mutant viruses were then generated via reverse genetics. Rescued mutant viruses bear the NA gene from CA/04 and the remaining 6 remaining 6 gene segments from A/Puerto Rico/8/1934(H1N1) (PR8). After three passages, a total of 189 mutant viruses bearing unique amino acid substitutions with different biochemical properties were generated (FIGS. 7A-7C). Of these mutant viruses, 96 had substitutions within the 119-190 region, 15 within the 190-241 region, and 78 within both regions of the HA. The positions of the mutations overlapped with the reported HA 116 antibody-binding sites (ABS) Sa (n=11), Sb (n=9), Ca1 (n=7), and Ca2 (n=7). There were 80 of the mutant viruses with a single substitution, 80 with two substitutions, and 29 with three or more substitutions. Interestingly, all the substitutions in rescued viruses were located outside of the RBS and do not directly contact the receptor molecule (FIG. 2A). In contrast, most substitutions within the highly conserved RBS failed to produce a viable virus via reverse genetics.

### Most Mutants do not Alter Antigenic Properties.

To determine the antigenic properties of mutant viruses generated, HAI assays were performed using ferret antisera. Out of 189 mutant viruses, only 5 mutants had significant changes in their antigenic properties by showing a ≥4-fold reduction in their HAI titers compared to WT. These 5 antigenically distinct mutants had at least one substitution in the HA ABS, with other substitutions mostly present within or close to the Ca1, Ca2, Sa, or Sb. Of note, the ferret sera generated against WT CA/04 were unable to neutralize the triple mutant D131 E-S193T-A198S. The serological data of the 189 mutants was then integrated with archived public data for seasonal H1 N1 (1977-2009) and 2009 H1 N1 viruses (2009-2016) and applied MAIVeSS to identify residues associated with the antigenicity. Results showed that 30 residues were associated with the antigenicity of H1 N1 IAV (FIGS. 7A-7C), and most of these residues were located within or close to the ABS, particularly Ca1, Ca2, Sa, and Sb (FIG. 2B).

### Substitutions Near the HA Receptor Binding Pocket can Result in High-Yield Traits in Both Cells and Eggs.

The substitutions were then assessed for how they affected virus yield in both cells and eggs by measuring the TCID50 titers for each mutant. 14 HY^{cell} mutants were identified that showed at least a 10-fold increase in virus yield compared to WT, as well as 29 LY^{ce11} mutants that showed at least a 10-fold decrease (FIG. 8).

The highest yield was observed in the N159D-K1661 mutant, with a yield of 1.52×10⁷ TCID50/mL, which was about 100-fold higher than WT. Additionally, 33 HY^{egg} mutants and 19 LY^{egg} mutants were identified when compared to WT. The D131E-S193T-A198S, N159D-K1661, and 1169F-D225G mutants had the highest titers in eggs, and were approximately 800-fold higher than WT. Of note, these three mutants also exhibited high-yield traits in cells, thus designated as HY^{both}. MAIVeSS was utilized to identify substitutions at 38 residues that were associated with virus yield. The majority of these residues were located on the surface of the HA trimer and in close proximity to the RBS pocket but distant from the pocket center (FIG. 2C). Interestingly, it was observed that certain substitutions with different amino acids could lead to different outcomes. For example, a change from small polar amino acids to nonpolar amino acids at residue 142 was predicted to enhance virus yield, whereas substitution to polar/charged amino acids at the same position was predicted to reduce virus yield in eggs and cells (Table 2).

Diversified glycan binding facilitates virus replication in cells and eggs. To investigate if high-yield trait correlates with glycan substructure binding properties, the receptor-binding properties of the 189 mutant viruses were analyzed using glycan microarrays comprising 75 glycoforms (FIG. 9). The binding signals to these glycan isoforms varied widely among the mutants (FIG. 8). Notably, all mutants exhibited strong binding avidity to glycans that were terminated with SA2,6Gal, as was expected.

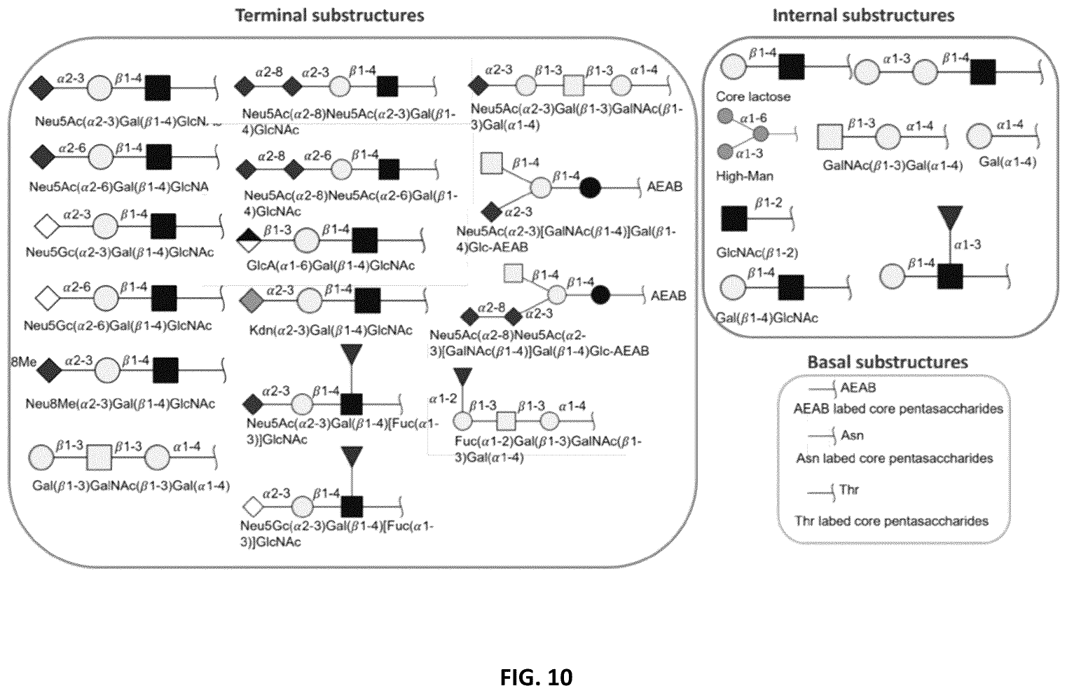

A matrix of 28 glycan substructure features was further used to group the glycans based on their internal and terminal substructures as well as their linkers (FIG. 10). Analysis revealed that HY^{c}e° mutants displayed elevated binding avidities to glycans terminated with Neu5Acα2-6Galβ1-4GlcNAc (6′SLN), but not to Neu5Acα2-3Galβ1-4GlcNAc (3′SLN), Neu5Aca2-3Galβ1-4(Fuca1-3)GlcNAc (sLeX) or Neu5Gcα2-3Galβ1-4GlcNAc. In contrast, HY^{egg} and HY^{both} mutants (such as D131E-S193T-A198S and 1169F-D225G) showed increased binding affinities to glycans terminated with 3′SLN and sLeX. Interestingly, a few HY^{egg} and HY^{bot}h mutants also exhibited significant increases in binding avidities to a glycan that is terminated with Neu5Gcα2-6Galβ1-4GlcNAc or Neu5Gcα2-3Galβ1-4GlcNAc.

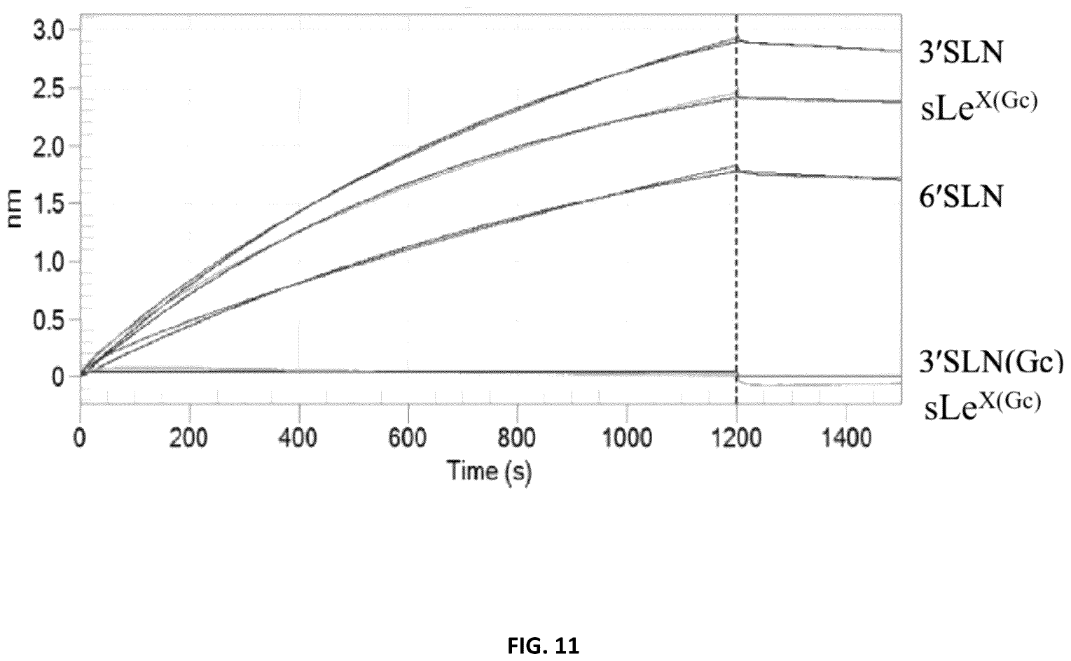

By employing biolayer interferometry analyses (BLI) for glycan binding profiling, the broadened binding specificity of the HY^{both} mutant D131 E-S193T-A198S was confirmed. Specifically, it was demonstrated that this mutant not only binds to 6′SLN, but also to 3′SLN and sLeX (FIG. 11).

MAIVeSS was used to identify the glycan substructures associated with yield traits in cells and eggs and the amino acid substitutions associated with binding preference to these glycan substructures. Analysis revealed several glycan terminal substructures that were significantly associated with high-yield traits, including 6′SLN, 3′SLN, sLeX, and Neu5Gcα2-6Galβ1-4GlcNAc. Additionally, it was found that certain internal substructures, such as core lactose, GlcNAcb1-2, and Galα1-4Galβ1-4GlcNAc, had a significant impact on glycan binding.

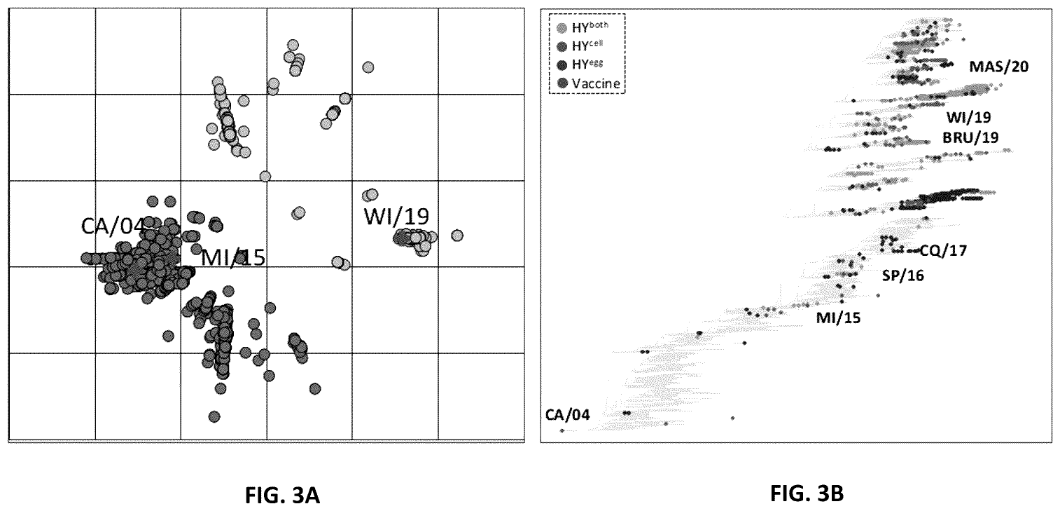

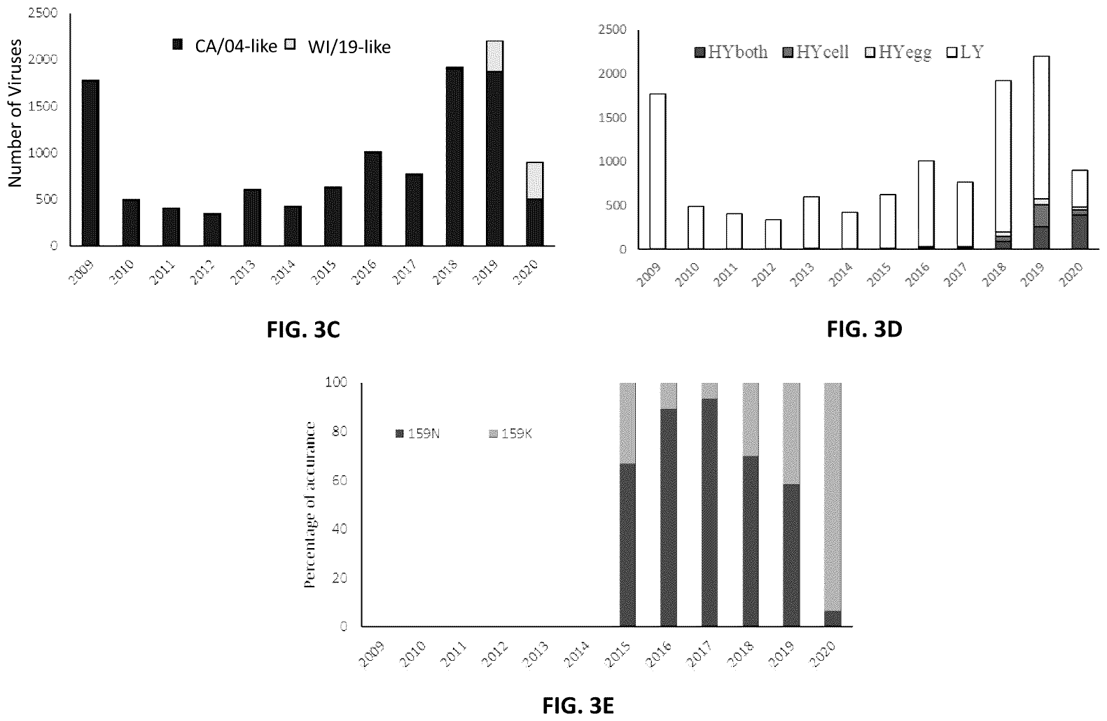

A subset of antigenically matched A(H1N1)pdm09 epidemic viruses were high-yield in both cells and eggs. MAIVeSS was used to assess both yield and antigenic properties for A(H1N1)pdm09 (2009-2020, n=11,424) in eggs and cells, in comparison to WT CA/04. Using the antigenic distance matrix generated by MAIVeSS, a sequence-based antigenic cartography was used, which revealed two antigenic clusters, CA/09 and WI/19 (FIG. 3A). Sequence analysis further demonstrated that the acquisition of N159K at ABS Sa resulted in an antigenic drift of HA of A/Wisconsin/588/2019(H1 N1) (WI/19) from that of CA/09.

Using MAIVeSS as the prediction tool, a total of 155 virus variants were identified as potential high-yield strains in eggs, 433 in cells, and 761 for both. Among those high-yield strains for both eggs and cells, 294 were CA/09-like viruses (38.6%), while 467 were WI/19-like viruses (61.4%). These high-yield strains were not geographically clustered and were scattered sporadically across the phylogenetic trees, without clear association with any particular lineages (FIG. 3B). However, the number of HY^{both} strains increased significantly after the emergence of WI/19-like variants (FIGS. 3C and 3D). Specifically, 256 out of 2,198 (11.65%) viruses in 2019 and 386 out of 895 (43.13%) viruses in 2020 were estimated to be HY^{both} strains. MAIVeSS analysis showed that the vaccine strain WI/19 has, compared to CA/04, an estimated increase of approximately 105-fold and 23-fold in virus yield in cell and eggs, respectively. Notably, WHO recommended WI/19 as cell-grown vaccine strain without additional engineering.

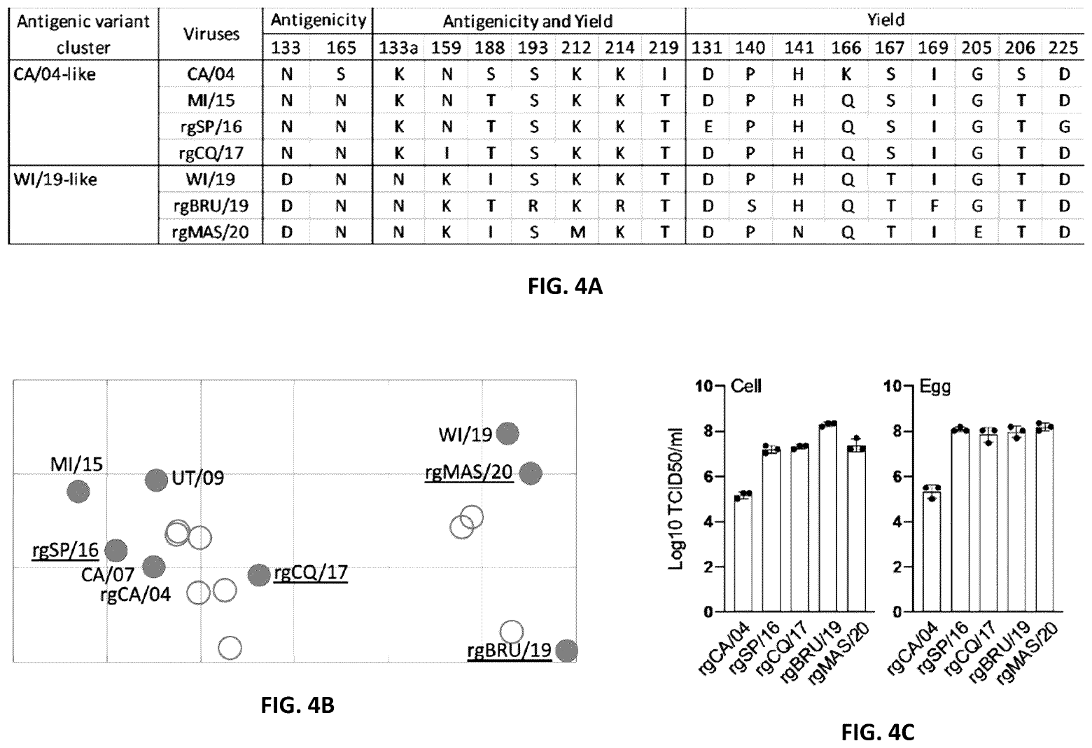

Multiple amino acid substitutions associated with yield properties were observed in these HY^{both} strains, but there were no consistent patterns observed across influenza seasons. However, after the 2018-2019 influenza season, viruses with K133aN, N159K/D/S, K166Q, S206T, and/or K214R were more likely to be high-yield strains (FIG. 4A).

To further validate the model, HA and NA genes for 4 predicted vaccine candidates were synthesized, and subsequently generated 4 reassortant viruses (i.e. rgSP/16, rgCQ/17, rgBRU/19 and rgMAS/20) with PR8 as the backbone and determined their antigenic and yield phenotypes. Antigenically, 2 viruses were shown as CA/04-like and the other 2 as WI/19-like (FIG. 4B), and all 4 vaccine candidates exhibited viral titers of >108 TCID50/mL in both eggs and cells, which were at least 100-fold higher than WT CA/04 (FIG. 4C). These results corroborated the prediction of their model.

Taken together, the findings indicate that the high-yield trait of A(H1N1)pdm09 viruses was sporadically distributed across different antigenic clusters and has become more prevalent since 2018. These experimental results confirm MAIVeSS's ability to identify antigenic matches and high-yield vaccine strains for A(H1 N1)pdm09 viruses.

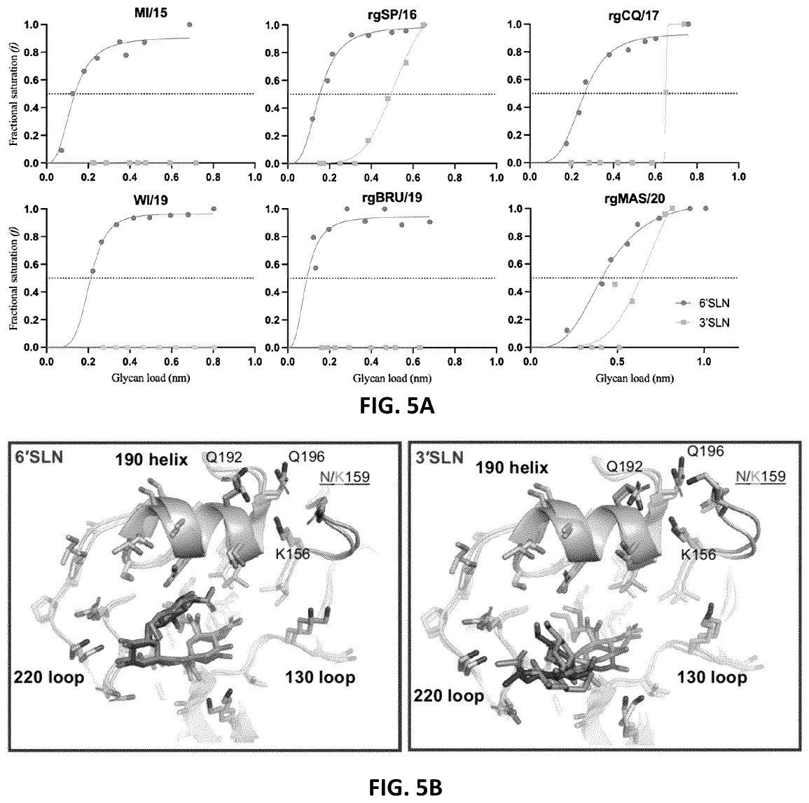

Diversifying influenza virus glycan binding profile facilitates the acquisition of high-yield properties. It was hypothesized that A(H1 N1)pdm09 acquired high-yield properties by binding to additional sialylated glycan receptors, particularly SA2-3Gal, or by increasing their glycan binding avidity to SA2-6Gal. To test this, BLI was conducted for 6 H1 N1 variants, including low-yield MI/15 and high-yield WI/19, as well as 4 high-yield vaccine candidates predicted by MAIVeSS. Results showed that 3 vaccine candidates rgSP/16, rgCQ/17 and rgMAS/20 bound to both 3′SLN and 6′SLN, whereas MI/15, WI/19, and one vaccine candidate rgBRU/19 bound only to 6′SLN. Furthermore, it was found that rgBRU/19 had a higher binding avidity to 6′SLN than MI/15 (FIG. 5A).

Among WI/19 and the 4 high-yield vaccine candidates, only about half of the residues linked to yield traits were conserved (FIG. 4A). However, N159K, K166Q, and S206T were consistently present in most of the naturally occurring high-yield strains and high-yield mutants from a mutagenesis study.

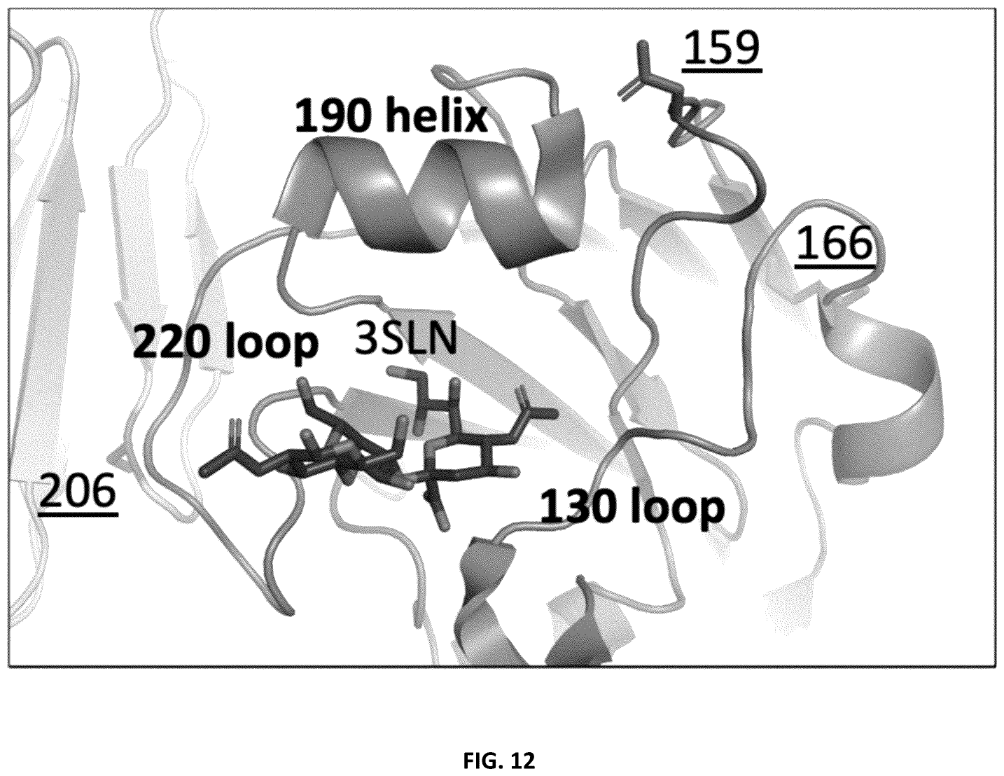

The effect of N159K, K166Q, and S206T on glycan binding affinity was investigated by conducting structural modeling based on the crystal structure of CA/04 HA complexed with 6′SLN and 3′SLN (FIG. 5B). In both complex structures, N159 was substituted with K, and energy minimization was performed to detect any possible allosteric structural changes that could affect ligand binding. In the HA:3′SLN structure, the sidechain of K159 flips toward the 190-helix, forming hydrogen bonds with the sidechains of both Q196 and Q192. This could cause a shift or tilt in the orientation of the 190-helix, resulting in a more compact receptor binding pocket and stronger binding with 3′SLN. In contrast, 6′SLN in the HA:6′SLN complex closely interacts with the 190-helix even in the original CA/04 structure, so the K159 mutation does not significantly enhance the binding of 6′SLN binding to HA. Additionally, K166Q may impact the conformation of the 130-loop, while S206T substitution has the potential to modify the structural conformation of the 220-loop (FIG. 12), thus affecting the binding of HA to glycan receptors. Therefore, modeling analysis supported that these three substitutions in HA can substantially increase the binding affinity of 3′SLN without major impact on the binding of 6′SLN.

In summary, diversity at the HA RBS of A(H1 N1)pdm09 can enhance virus yields in both cell and egg substrates by increasing sialylated glycan binding avidity or diversifying virus binding to different sialylated glycan receptors.

In this Example, MAIVeSS was developed, a machine learning based framework, that can accurately predict both antigenicity and yield phenotypes based on HA protein sequences. The training dataset consisted of a library of 189 mutant viruses generated by epPCR-based reverse genetics targeting residues 126-244 (H3 numbering). It was observed that acquisition of N159K, a key marker for antigenic drift according to the model described herein, led to changes in antigenicity from CA/09 to WI/19, consistent with published reports and facilitated acquisition of the high-yield trait in a significant proportion of A(H1 N1)pdm09 epidemic strains during recent influenza seasons. While the model described here focuses on HA, it is important to note that antigenic drift of neuraminidase (NA) has also been well-documented in H1 N1 and H3N2 influenza viruses.

As such, ongoing efforts are aimed to expand MAIVeSS prediction capacity to include both HA and NA proteins. The glycan profiling analysis conducted on 43 high-yield mutants suggested that diversifying glycan binding profiles could enhance virus replication in both eggs and cells. Specifically, increased binding avidities to SA2-6Gal results in higher virus yield in cells, while broadening glycan binding capabilities to SA2-3Gal or sLeX improves virus yield in eggs. Studies indicate that a small subset of A(H1N1)pdm09 epidemic viruses naturally prefer both SA2-6Gal and SA2-3Gal, allowing them to replicate efficiently in both cells and eggs without adaptation. On the other hand, some high-yield strains (e.g. WI/19) were found to have no significant changes in binding preference to either 3′SLN or 6′SLN (FIG. 5A), indicating that other glycan substructures present in eggs and/or cells may be involved in the high-yield trait for these epidemic viruses. Further studies are needed to investigate this possibility.

Both SA2-6Gal and SA2-3Gal receptors are expressed in MDCK cells and chicken embryonated eggs. However, SA2-3Gal receptors are predominantly expressed in eggs while MDCK cells contain a similar amount of SA2-6Gal and SA2-3Gal. In addition to SA2-3Gal and SA2-6Gal, neutral glycans such as high-mannose glycans and glycans terminated with Gal and GalcNAc are also commonly found in eggs. Mass spectrometry analyses showed some glycans in eggs are fucosylated. The CA/04, the prototype A(H1N1)pdm09 virus which showed poor replication in both MDCK cells and eggs, had a strong binding preference for SA2-6Gal and did not bind to SA2-3Gal. In humans, there is no selection pressure for either cell-based or egg-based replication efficiency. Thus, findings suggested that ad hoc substitutions at the HA RBS across A(H1 N1)pdm09 strains likely enabled a subset of these variants to expand their binding preference from SA2-6Gal to both SA2-6Gal and SA2-3Gal, resulting in the acquisition of a high-yield trait. This Example demonstrates that it is possible to select naturally circulating strains as vaccine candidates without the need for further engineering.

In summary, the data from the proof-of-concept experiments in this Example confirmed that MAIVeSS enables rapid selection of antigenically matching and high-yield influenza strains directly from clinical isolates as potential seed viruses to accelerate vaccine production and facilitate timely supply of seasonal vaccines.

### Validation of Glycan Binding Profile for D131 E-S193T-A198S Using Biolayer Interferometry Analyses

To confirm the binding avidities observed in the glycan microarray, biolayer interferometry was used to analyze the binding of the HY^{both} mutant D131 E-S193T-A198S to five representative glycan analogs: Neu5Acα2-3Galβ1-4GlcNAcβ (3′SLN), Neu5Acα2-6Gal31-4GlcNAc3 (6′SLN), Neu5Acα2-3Gal31-4[Fucα1-3]GlcNAcβ (sLeX), Neu5Gcα2-3Gal31-4GlcNAcβ (3′SLN(Gc)), and Neu5Gcα2-3Galβ1-4[Fucα1-3]GlcNAcp (sLeX(Gc)). The HYboth mutant had broadened binding avidities from 6′SLN to 3′SLN and sLeX whereas WT CA/04 did not bind to 3′SLN and sLeX. The mutant had a 1.61-fold lower binding avidity to 6′SLN than to 3′SLN, and it did not bind to 3′SLN(Gc) or sLeX(Gc), similar to WT CA/04. These results were consistent with those obtained from the glycan microarray.

### Genomic Sequencing

Viral RNA was isolated from 200 μl of the sample using a 5X MagMAXTM Pathogen RNA/DNA kit (Thermo Fisher Scientific, Pittsburgh, PA) according to the manufacturer's instructions, and a total of 80 μl RNA was obtained. cDNA synthesis was carried out using SuperScript III Reverse Transcriptase (Invitrogen, Grand Island, NY) with 10 μl of the isolated RNA and the influenza virus-specific primer Uni12 (5′-AGCAAAAGCAGG-3′; SEQ ID NO:6)), with a total reaction volume of 25 μl. The HA segment of mutants was amplified using the Phusion High-Fidelity PCR Kit (Thermo Fisher Scientific, Pittsburgh, PA) and the primers CA/04_HA_F (5′-ATGAAGGCAATACTAGTAGTTCTGC-3′; SEQ ID NO:7) and CA/04_HA_R (5′-TTAAATACATATTCTACACTGTAGAGACC-3′; SEQ ID NO:8). The PCR products (50 μl) were purified using the GeneJET PCR Purification kit (Thermo Fisher Scientific, Pittsburgh, PA) as per the manufacturer's instructions. The HA sequences of the mutants were confirmed by Sanger sequencing at Eurofins (Louisville, KY) or University of Missouri DNA core.

### Generation of Ferret Antisera

Ferret antisera were produced in male or female ferrets aged 6 to 8 weeks, which were confirmed to be seronegative for CA/04, A/Switzerland/9715293/2013 (H3N2), and A/Hong Kong/4801/2014(H3N2). Each ferret was intranasally inoculated with 106 TCID50 of either the wild-type virus or a mutant virus to be tested. Ferret sera were collected 21 days after inoculation and used for antigenic phenotyping through serological assays.

### Model Comparison and Parameter Optimization

To ensure the robustness of the analyses, the performance of the sparse learning model was compared with three other commonly used sparse models: LASSO, RIDGE, and SGL. Additionally, the model was also compared with two other sparse learning methods, the L1- and L2-norm regularized method and the L1- and L∞-norm Composite Absolute Penalties method (iCAP). LASSO uses L1-norm regularization, RIDGE uses L2-norm regularization, SGL uses group Lasso regularization, L1- and L2-norm regularization combines L1-norm and L2-norm regularization, and iCAP combines L1-norm and L∞-norm regularization. The performance of these models was evaluated based on various metrics, such as accuracy, Root Mean Square Error (RMSE), and predictive power. Briefly, the LASSO regression seeks to minimize the following: ∥y−Xw∥₂+λ₁∥w∥₁, the RIDGE regression seeks to minimize the following: ∥y−Xw∥₂+λ₁∥w∥₂, the RIDGE regression seeks to minimize the following: ∥y−Xw∥₂+λ₁Σₜ∥wₗ∥₂+λ₂∥w∥₁, the L1- and L2-norm regularized method seeks to minimize: ∥y−XW∥₂+λ1 Σ∥[∥W_{G}_{₁}∥γ₁, ∥W_{G}_{₂}∥γ₂, . . . , ∥W_{G}_{ₙ}∥γₙ,]∥γ₀, and the iCAP seeks to minimize: ∥y−XW∥₂+λ₁Σₗ∥[∥W_{G}_{₁}∥γ₁, ∥W_{G}_{₂}∥γ₂, . . . , ∥W_{G}_{ₙ}∥γₙ,]∥γ₀,where y is the vector of actual response value, w is the vector of weights, X is the matrix of explanatory value, λ₁ is constraint parameters, and ∥⋅∥1 is the L1-norm, ∥⋅∥2 is the L2-norm, Gₙ's, n=1, . . . , N is indices of n-th pre-defined group, W_{G}_{ₙ} is corresponding vector of weight, ∥⋅∥|γₙ is group norm, and ∥⋅∥γ₀ is overall norm. Here γ₀ equals 1 was chosen as the overall norm. If γ₁=γ₂= . . . =γ_{N}∞ were chosen as group norm, which is referred to as the algorithm iCAP. If γ₁=γ₂= . . . =γ_{N}=2 were chosen as group norm, which is referred to the L1- and L2-norm regularized method.

The PIMA (Protein-Protein Interactions in Macromolecular Analysis) method was utilized to incorporate the biochemical properties of amino acids. PIMA assigns the 20 amino acids into nine groups and assigns a different numerical code for different mutations. Substitutions between different pairs of residues are given an inclusive weight between 0 and 5. The weights assigned to each feature in the learning results indicate the significance of the feature, with greater weight indicating higher significance.

To investigate the impact of amino acid substitutions on growth phenotype, a three-group method was utilized for assigning amino acids to different groups based on their biophysical properties. Specifically, each amino acid was classified into one of three groups: nonpolar (including V, L, I, M, C, F, W, and Y), small nonpolar (including G, A, and P), and polar/charged (including S, T, N, Q, H, D, E, K, and R) 6. Using this classification, if a mutation occurred between two groups at a given residue j (e.g., nonpolar to small polar), the j-th element of the feature vector xi was set to 1; otherwise, it was set to 0. This approach allows one to evaluate the directionality of amino acid substitutions on the growth phenotype.

The regularization parameters of the sparse learning model were optimized using root mean square error (RMSE). The choice of regularization methods (LASSO, RIDGE, SGL, L1- and L2-norm, or iCAP) and the scoring method were also based on RMSE, determined through 10-fold cross-validation. In this method, 90% of the data were used for training and 10% for testing, and the model's performance was evaluated based on the RMSE, with smaller values indicating better performance. RMSE was defined as:

$$RMSE = \sqrt{\frac{1}{n} {\sum }_{i = 1}^{n} {( {\hat{y}}_{i} - {y}_{i} )}^{2}}$$

To evaluate the performance of the method, a comparison based on accuracy was performed. Specifically, a threshold of 4-fold (2 units of antigenic distance) was considered to determine if two viruses were antigenically distinct and exhibited antigenic drift. Using this threshold, classification tasks were defined to measure the prediction accuracy. The accuracy metric describes the proportion of correctly predicted results among the total number of samples.

### Bootstrapping Analyses

To evaluate the reliability of the selected features by MTL-GGSL, 100 independent experiments were conducted with 80% of the training data in each experiment. Only features with a bootstrap value of at least 80 across multiple tasks were retained, resulting in a set of unique features that were chosen as the final features learned by MTL-GGSL.

### Related Machine Learning Methods

Over the past few years, several computational models have been developed to identify influenza antigenic variants using genomic sequences. These models include sparse learning, bivariate correlation analysis, Bayesian model, naïve Bayes classifier, random forest, regression models, decision tree algorithms, and convolutional neural network model. Among these models, sparse learning has proven to be efficient and generalizable in identifying the association between residues and antigenicity of multiple subtypes of IAVs, including H1 N1, H3N2, and H5N1. Additionally, generalized hierarchical sparse models have been used to identify the synergistic effects of multiple amino acid substitutions on antigenic changes. To overcome the challenges in data integration, multi-task machine learning was developed, which assigns datasets to individual tasks and considers the relationship between different tasks. In another study, group Least Absolute Shrinkage and Selection Operator (LASSO) was developed to accommodate multiple types of features and explore the relationships between different feature groups. Although these models have proven effective in identifying antigenic variants, none of them have considered virus yield. Therefore, they cannot be used to directly identify antigenic match and high-yield viruses that can be produced readily based on genetic sequences.

In view of the above, it will be seen that the several objects and advantages of the present disclosure have been achieved and other advantageous results have been obtained.

As various changes could be made in the above constructions without departing from the scope of the invention, it is intended that all matter contained in the above description or shown in the accompanying drawings shall be interpreted as illustrative and not in a limiting sense.

What is claimed is:

1. A method for identifying one or more preferred viral strains for vaccine development, said method comprising:
sequencing hemagglutinin present in one or more circulating viral strains to generate one or more circulating hemagglutinin sequences;
sequencing hemagglutinin present in each of one or more candidate viral strains to generate one or more candidate hemagglutinin sequences;
providing an input to a machine learning algorithm, wherein the input comprises the one or more circulating hemagglutinin sequences and the one or more candidate hemagglutinin sequences;
using the machine learning algorithm to predict one or more desired phenotypes selected from the group consisting of:
antigenic difference values between each of the one or more circulating viral strains and each of the one or more candidate viral strains; and
an egg yield value, a cell yield value, and a combined yield value for each of the one or more candidate viral strains;
identifying one or more preferred viral strains from among the one or more candidate viral strains by identifying each of the one or more candidate viral strains that are predicted to have one or more desired phenotypes selected from the group consisting of:
low antigenic difference values; and
a high egg yield value, cell yield value, or combined yield value.

2. The method of claim 1, wherein the antigenic difference values are predicted by assessing differences between amino acid residues in the circulating hemagglutinin sequences and in the one or more candidate hemagglutinin sequences, wherein locations of the amino acid residues consist of positions 45, 46, 53, 60, 63, 65, 96, 125c, 129, 133, 133a, 142, 144, 158, 159, 160, 165, 188, 189, 193, 194, 198, 208, 212, 214, 219, 271, 273, 274 and 290.

3. The method of claim 2, wherein the locations of the amino acid residues consist of positions 53, 60, 63, 125c, 133, 158, 159, 212 and 274.

4. The method of claim 3, wherein the locations of the amino acid residues consist of positions 53, 60, 133, 159 and 274.

5. The method of claim 1, wherein the egg yield value, the cell yield value, and the combined yield value for each of the one or more candidate viral strains are predicted assessing differences between yield amino acid residues in the circulating hemagglutinin sequences and in the one or more candidate hemagglutinin sequences, wherein locations of the yield amino acid residues consist of positions 126, 131, 132, 133a, 137, 138, 140, 141, 142, 144, 146, 149, 157, 159, 162, 166, 167, 169, 173, 174, 178, 182, 188, 189, 192, 193, 197, 198, 205, 206, 211, 212, 214, 219, 222, 225, 229 and 237.

6. The method of claim 5, wherein the locations of the yield amino acid residues consist of positions 126, 132, 133a, 138, 141, 142, 144, 157, 159, 166, 167, 169, 182, 188, 189, 193, 198, 205, 214 and 225.

7. The method of claim 5, wherein the locations of the yield amino acid residues consist of positions 126, 133a, 138, 141, 142, 144, 157, 159, 166, 169, 182, 188, 193, 205 and 214.

8. The method of claim 5, wherein the locations of the yield amino acid residues consist of positions 126, 133a, 166, 169, 182, 193, 214.
